(19) United States
(12) Patent Application Publication
(10) Pub. No.: US 2010/0323426 A1
(43) Pub. Date: Dec. 23, 2010

(54) Heterologous and Homologous Cellulase Expression System

(76) Inventors: Benjamin S. Bower, Palo Alto, CA (US); Edmund A. Larenas, Palo Alto, CA (US)

Correspondence Address: DANISCO US INC.;ATTENTION: LEGAL DEPARTMENT, 925 PAGE MILL ROAD, PALO ALTO, CA, 94304, US

(21) Appl. No.: 12/602,963

(22) PCT Filed: Jun. 5, 2008
(86) PCT No.: PCT/US08/07077

§ 371 Date: Aug. 30, 2010

Related U.S. Application Data
(60) Provisional application No. 60/933,894, filed on Jun. 8, 2007.

(51) Int. Cl. C12N 9/42; C12N 1/15; C12N 9/24

(52) U.S. Cl. 435/209; 435/254.11; 435/254.9; 435/254.8; 435/254.7; 435/254.6; 435/254.5; 435/254.4; 435/254.3; 435/200

(57) ABSTRACT

The present invention provides filamentous fungi that express a combination of heterologous and homologous polypeptides, polypeptide mixtures comprising a combination of heterologous and homologous polypeptides and methods of producing the polypeptide mixtures.

### CROSS-REFERENCES TO RELATED APPLICATION

The present application claims benefit of and priority to U.S. Provisional Application Ser. No. U.S. 60/933,894, filed Jun. 8, 2007, which is incorporated herein by reference in its entirety.

### STATEMENT REGARDING FEDERALLY SPONSORED RESEARCH

Portions of this work were funded by Subcontract No. ZC0-0-30017-01 with the National Renewable Energy Laboratory under Prime Contract No. DE-AC36-99G010337 with the United States Department of Energy. Accordingly, the United States Government may have certain rights in the invention.

### INTRODUCTION

Cellulose and hemicellulose are the most abundant plant materials produced by photosynthesis. They can be degraded and used as an energy source by numerous microorganisms, including bacteria, yeast and fungi, which produce extracellular enzymes capable of hydrolyzing the polymeric substrates to monomeric sugars. Cellulases are enzymes that hydrolyze cellulose (beta-1,4-glucan or beta D-glucosidic linkages) resulting in the formation of glucose, cellobiose, cellooligosaccharides, and the like. Cellulases have been traditionally divided into three major classes: endoglucanases (EC 3.2.1.4) (“EG”), exoglucanases or cellobiohydrolases (EC 3.2.1.91) (“CBH”) and beta-glucosidases ([beta]-D-glucoside glucohydrolase; EC 3.2.1.21) (“BG”). Endoglucanases act mainly on the amorphous parts of the cellulose fiber, whereas cellobiohydrolases are also able to degrade crystalline cellulose. In order to efficiently convert crystalline cellulose to glucose the complete cellulase system comprising components from each of the CBH, EG and BG classifications is required, with isolated components less effective in hydrolyzing crystalline cellulose (Filho et al., Can. J. Microbiol. 42:1-5, 1996). It would be advantageous to express these multi-component cellulase systems cellulases in a filamentous fungus for industrial scale cellulase production.

### SUMMARY

Accordingly, the present teachings provide filamentous fungi that express a combination of heterologous and homologous polypeptides, polypeptide mixtures comprising a combination of heterologous and homologous polypeptides and methods of producing the polypeptide mixtures.

In some embodiments, the present teachings provide a filamentous fungus comprising two or more polynucleotides that encode two or more heterologous polypeptides and a polynucleotide encoding a homologous polypeptide. The filamentous fungus is capable of expressing the heterologous and homologous polypeptides that together form a functional mixture.

In some embodiments, the present teachings provide a culture medium comprising a population of the filamentous fungus of the present teachings.

In some embodiments, the present teachings provide a polypeptide mixture comprising two or more heterologous polypeptides and a homologous polypeptide. The polypeptide mixture can be obtained from the filamentous fungi of the present teachings.

In some embodiments, the present teachings provide a method of producing a mixture of cellulases. The method comprises obtaining a polypeptide mixture comprising two or more heterologous polypeptides and a homologous polypeptide from the filamentous fungus of the present teachings. In some embodiments, the heterologous polypeptides are an exo-cellobiohydrolase and an endoglucanase, and the homologous polypeptide is an exo-cellobiohydrolase. The heterologous exo-cellobiohydrolase and the homologous exo-cellobiohydrolase, may, but need not be the same member of exo-cellobiohydrolases.

These and other features of the present teachings are set forth below.

### BRIEF DESCRIPTION OF THE FIGURES

The skilled artisan will understand that the drawings are for illustration purposes only. The drawings are not intended to limit the scope of the present teachings in any way.

FIG. 1 provides the nucleotide sequence (SEQ ID NO: 1) of the heterologous cellulase fusion construct comprising 2656 bases.

FIG. 2 provides the predicted amino acid sequence (SEQ ID NO: 2) of the cellulase fusion protein based on the nucleic acid sequence of FIG. 1.

FIGS. 3A-F depicts the nucleotide sequence (SEQ ID NO:14) of the pTrex4 vector containing the E1 catalytic domain.

FIG. 4 depicts the plasmid map of T. reesei expression vector pTrex3g.

FIG. 5A depicts the expression vector pTrex3g-Hgrisea-cbh1 used for making an exemplary tripartite strain.

FIGS. 5B-E provides the nucleotide sequence (SEQ ID NO: 7) of the expression vector of FIG. 5A.

FIG. 6 shows the three DNA expression fragments transformed into the cbh1 deleted strain to create a 4-part strain.

FIG. 7A provides the nucleotide sequence (SEQ ID NO: 8) from start to stop codon of the polynucleotide expressing the engineered CBHI protein.

FIG. 7B provides the sequence of the engineered CBHI protein (SEQ ID NO: 9). The CBHI signal sequence is underlined.

FIG. 8A depicts the cbhI expression vector pTrex3g-cbh1.

FIGS. 8B-F provides the nucleotide sequence (SEQ ID NO: 10) of the expression vector pTrex3g-cbh1.

FIG. 9A provides the nucleotide sequence (SEQ ID NO: 11) from start to stop codon of the polynucleotide expressing the engineered CBHI protein.

FIG. 9B provides the amino acid sequence of the engineered CBHII protein (SEQ ID NO: 12). The signal sequence is underlined).

FIG. 10A depicts the cbhII expression vector pExp-cbhII.

FIGS. 10B-G provides the nucleotide sequence (SEQ ID NO: 13) of the expression vector pExp-cbhII.

### DETAILED DESCRIPTION OF VARIOUS EMBODIMENTS

The present teachings will now be described in detail by way of reference only using the following definitions and examples. Unless defined otherwise herein, all technical and scientific terms used herein have the same meaning as commonly understood by one of ordinary skill in the art to which this invention belongs. Numeric ranges are inclusive of the numbers defining the range. The headings provided herein are not limitations of the various aspects or embodiments which can be had by reference to the specification as a whole. Accordingly, the terms defined immediately below are more fully defined by reference to the specification as a whole.

The term “polypeptide” as used herein refers to a compound made up of a single chain of amino acid residues linked by peptide bonds. The term “protein” as used herein is used interchangeably with the term “polypeptide.”

The term “nucleic acid” and “polynucleotide” are used interchangeably and encompass DNA, RNA, cDNA, single stranded or double stranded and chemical modifications thereof. Because the genetic code is degenerate, more than one codon may be used to encode a particular amino acid, and the present invention encompasses all polynucleotides, which encode a particular amino acid sequence.

The term “recombinant” when used in reference to a cell, nucleic acid, protein or vector, indicates that the cell, nucleic acid, protein or vector, has been modified by the introduction of a heterologous nucleic acid or protein or the alteration of a native nucleic acid or protein, or that the cell is derived from a cell so modified or that a protein is expressed in a non-native or genetically modified environment, e.g., in an expression vector for a prokaryotic or eukaryotic system. Thus, for example, recombinant cells express nucleic acids or polypeptides that are not found within the native (non-recombinant) form of the cell or express native genes that are otherwise abnormally expressed, under expressed, over expressed or not expressed at all.

The term “heterologous” with reference to a polynucleotide or polypeptide refers to a polynucleotide or polypeptide having a sequence that does not naturally occur in a host cell. In some embodiments, the polypeptide is a commercially important industrial protein and in some embodiments, the heterologous polypeptide is a therapeutic protein. It is intended that the term encompasses proteins that are encoded by naturally occurring genes, mutated genes, and/or synthetic genes.

The term “homologous” with reference to a polynucleotide or polypeptide refers to a polynucleotide or polypeptide having a sequence that occurs naturally in the host cell.

As used herein, a “fusion nucleic acid” comprises two or more nucleic acids operably linked together. The nucleic acid may be DNA, both genomic and cDNA, or RNA, or a hybrid of RNA and DNA. Nucleic acid encoding all or part of the sequence of a polypeptide can be used in the construction of the fusion nucleic acid sequences. In some embodiments, nucleic acid encoding full length polypeptides are used. In some embodiments, nucleic acid encoding a portion of the polypeptide may be employed.

The term “fusion polypeptide” refers to a protein that comprises at least two separate and distinct regions that may or may not originate from the same protein. For example, a signal peptide linked to the protein of interest wherein the signal peptide is not normally associated with the protein of interest would be termed a fusion polypeptide or fusion protein.

The terms “recovered”, “isolated”, and “separated” are used interchangeably herein to refer to a protein, cell, nucleic acid, amino acid etc. that is removed from at least one component with which it is naturally associated.

As used herein, the term “gene” refers to a polynucleotide (e.g., a DNA segment) involved in producing a polypeptide chain, that may or may not include regions preceding and following the coding region, e.g. 5′ untranslated (5′ UTR) or “leader” sequences and 3′ UTR or “trailer” sequences, as well as intervening sequences (introns) between individual coding segments (exons).

As used herein, the term “promoter” refers to a nucleic acid sequence that functions to direct transcription of a downstream gene. The promoter will generally be appropriate to the host cell in which the target gene is being expressed. The promoter together with other transcriptional and translational regulatory nucleic acid sequences (also termed “control sequences”) are necessary to express a given gene. In general, the transcriptional and translational regulatory sequences include, but are not limited to, promoter sequences, ribosomal binding sites, transcriptional start and stop sequences, translational start and stop sequences, and enhancer or activator sequences.

As used herein, the term “operably linked” means that the transcriptional nucleic acid is positioned relative to the coding sequences in such a manner that transcription is initiated. Generally, this will mean that the promoter and transcriptional initiation or start sequences are positioned 5′ to the coding region. The transcriptional nucleic acid will generally be appropriate to the host cell used to express the protein. Numerous types of appropriate expression vectors, and suitable regulatory sequences are known in the art for a variety of host cells.

As used herein, the term “expression” refers to the process by which a polypeptide is produced based on the nucleic acid sequence of a gene. The process includes both transcription and translation.

As used herein, the term “vector” refers to a polynucleotide construct designed to introduce nucleic acids into one or more cell types. Vectors include cloning vectors, expression vectors, shuttle vectors, plasmids, cassettes and the like.

As used herein, the term “expression vector” refers to a vector that has the ability to incorporate and express heterologous DNA fragment in a foreign cell. Many prokaryotic and eukaryotic expression vectors are commercially available.

As used herein, the terms “DNA construct,” “transforming DNA” and “expression vector” are used interchangeably to refer to DNA used to introduce sequences into a host cell or organism. The DNA may be generated in vitro by PCR or any other suitable technique(s) known to those in the art, for example using standard molecular biology methods described in Sambrook et al. In addition, the DNA of the expression construct could be artificially, for example, chemically synthesized. The DNA construct, transforming DNA or recombinant expression cassette can be incorporated into a plasmid, chromosome, extrachromosomal element, mitochondrial DNA, plastid DNA, virus, or nucleic acid fragment. Typically, the recombinant expression cassette portion of an expression vector, DNA construct or transforming DNA includes, among other sequences, a nucleic acid sequence to be transcribed and a promoter. In preferred embodiments, expression vectors have the ability to incorporate and express heterologous DNA fragments in a host cell.

The term “introduced” in the context of inserting a nucleic acid sequence into a cell, means “transfection” or “transformation” or “transduction” and includes reference to the incorporation of a nucleic acid sequence into a eukaryotic or prokaryotic cell where the nucleic acid sequence may be incorporated into the genome of the cell (for example, chromosome, extrachromosomal element, plasmid, plastid, or mitochondrial DNA), converted into an autonomous replicon, or transiently expressed (for example, transfected mRNA).

By the term “host cell” is meant a cell that contains a vector and supports the replication, and/or transcription or transcription and translation (expression) of the expression construct.

As used herein, the term “culturing” refers to growing a population of cells under suitable conditions in a liquid, semi-solid or solid medium.

As used herein, “substituted” and “modified” are used interchangeably and refer to a sequence, such as an amino acid sequence or a nucleic acid sequence that includes a deletion, insertion, replacement or interruption of a naturally occurring sequence. Often in the context of the invention, a substituted sequence shall refer, for example, to the replacement of a naturally occurring residue.

As used herein, “modified enzyme” refers to an enzyme that includes a deletion, insertion, replacement or interruption of a naturally occurring sequence.

The term “variant” refers to a region of a protein that contains one or more different amino acids as compared to a reference protein, for example, a naturally occurring or wild-type protein.

The term “cellulase” refers to a category of enzymes capable of hydrolyzing cellulose (beta-1,4-glucan or beta D-glucosidic linkages) polymers to shorter cello-oligosaccharide oligomers, cellobiose and/or glucose.

The term “exo-cellobiohydrolase” (CBH) refers to a group of cellulase enzymes classified as EC 3.2.1.91 and/or those in certain GH families, including, but not limited to, those in GH families 5, 6, 7, 9 or 48. These enzymes are also known as exoglucanases or cellobiohydrolases. CBH enzymes hydrolyze cellobiose from the reducing or non-reducing end of cellulose. In general a CBHI type enzyme preferentially hydrolyzes cellobiose from the reducing end of cellulose and a CBHII type enzyme preferentially hydrolyzes the non-reducing end of cellulose.

The term “cellobiohydrolase activity” is defined herein as a 1,4-D-glucan cellobiohydrolase activity which catalyzes the hydrolysis of 1,4-beta-D-glucosidic linkages in cellulose, cellotetriose, or any beta-1,4-linked glucose containing polymer, releasing cellobiose from the ends of the chain. As used herein, cellobiohydrolase activity is determined by release of water-soluble reducing sugar from cellulose as measured by the PHBAH method of Lever et al., 1972, Anal. Biochem. 47: 273-279. A distinction between the exoglucanase mode of attack of a cellobiohydrolase and the endoglucanase mode of attack is made by a similar measurement of reducing sugar release from substituted cellulose such as carboxymethyl cellulose or hydroxyethyl cellulose (Ghose, 1987, Pure & Appl. Chem. 59: 257-268). A true cellobiohydrolase will have a very high ratio of activity on unsubstituted versus substituted cellulose (Bailey et al, 1993, Biotechnol. Appl. Biochem. 17: 65-76).

The term “endoglucanase” (EG) refers to a group of cellulase enzymes classified as EC 3.2.1.4, and/or those in certain GH families, including, but not limited to, those in GH families 5, 6, 7, 8, 9, 12, 17, 31, 44, 45, 48, 51, 61, 64, 74 or 81. An EG enzyme hydrolyzes internal beta-1,4 glucosidic bonds of the cellulose. The term “endoglucanase” is defined herein as an endo-1,4-(1,3;1,4)-beta-D-glucan 4-glucanohydrolase which catalyses endohydrolysis of 1,4-beta-D-glycosidic linkages in cellulose, cellulose derivatives (for example, carboxy methyl cellulose), lichenin, beta-1,4 bonds in mixed beta-1,3 glucans such as cereal beta-D-glucans or xyloglucans, and other plant material containing cellulosic components. As used herein, endoglucanase activity is determined using carboxymethyl cellulose (CMC) hydrolysis according to the procedure of Ghose, 1987, Pure and Appl. Chem. 59: 257-268.

The term “beta-glucosidase” is defined herein as a beta-D-glucoside glucohydrolase classified as EC 3.2.1.21, and/or those in certain GH families, including, but not limited to, those in GH families 1, -3, 9 or 48, which catalyzes the hydrolysis of cellobiose with the release of beta-D-glucose. As used herein, beta-glucosidase activity may be measured by methods known in the art, e.g., HPLC.

“Cellulolytic activity” encompasses exoglucanase activity, endoglucanase activity or both types of enzyme activity, as well as beta-glucosidase activity.

The terms “thermally stable” and “thermostable” refer to polypeptides or enzymes of the present teaching that retain a specified amount of biological, e.g., enzymatic, activity after exposure to an elevated temperature, i.e., higher than room temperature. In some embodiments, a polypeptide or an enzyme is considered thermo stable if it retains greater than 50%, 60%, 70%, 75%, 80%, 85%, 90%, 95% or 98% of its biological activity after exposure to a specified temperature, e.g., 40° C., 45° C., 50° C., 55° C., 60° C., 65° C., 70° C., 75° C. or 80° C. for 2, 5, 7, 10, 15, 20, 30, 40, 50 or 60 minutes at a pH of, e.g., 4, 4.5, 5, 5.5, 6, 6.5, 7, 7.5 or 8.

The term “filamentous fungi” means any and all filamentous fungi recognized by those of skill in the art. In general, filamentous fungi are eukaryotic microorganisms and include all filamentous forms of the subdivision Eumycotina. These fungi are characterized by a vegetative mycelium with a cell wall composed of chitin, beta-glucan, and other complex polysaccharides. In some embodiments, the filamentous fungi of the present teachings are morphologically, physiologically, and genetically distinct from yeasts. In some embodiments, the filamentous fungi include, but are not limited to the following genera: Aspergillus, Acremonium, Aureobasidium, Beauveria, Cephalosporium, Ceriporiopsis, Chaetomium paecilomyces, Chrysosporium, Claviceps, Cochiobolus, Cryptococcus, Cyathus, Endothia, Endothia mucor, Fusarium, Gilocladium, Humicola, Magnaporthe, Myceliophthora, Myrothecium, Mucor, Neurospora, Phanerochaete, Podospora, Paecilomyces, Penicillium, Pyricularia, Rhizomucor, Rhizopus, Schizophylum, Stagonospora, Talaromyces, Trichoderma, Thermomyces, Thermoascus, Thielavia, Tolypocladium, Trichophyton, and Trametes pleurotus. In some embodiments, the filamentous fungi include, but are not limited to the following: A. nidulans, A. niger, A. awomari, e.g., NRRL 3112, ATCC 22342 (NRRL 3112), ATCC 44733, ATCC 14331 and strain UVK 143f, A. oryzae, e.g., ATCC 11490, N. crassa, Trichoderma reesei, e.g., NRRL 15709, ATCC 13631, 56764, 56765, 56466, 56767, and Trichoderma viride, e.g., ATCC 32098 and 32086.

The term “Trichoderma” or “Trichoderma species” used herein refers to any fungal organisms which have previously been classified as a Trichoderma species or strain, or which are currently classified as a Trichoderma species or strain, or as a Hypocrea species or strain. In some embodiments, the species include Trichoderma longibrachiatum, Trichoderma reesei, Trichoderma viride, or Hypocrea jecorina. Also contemplated for use as an original strain are cellulase-overproducing strains such as T. longibrachiatum/reesei RL-P37 (Sheir-Neiss et al., Appl. Microbiol. Biotechnology, 20 (1984) pp. 46-53; Montenecourt B. S., Can., 1-20, 1987), and Rut-C30 strain. In some embodiments, the production of cellulases in the species targeted for improvement is tightly regulated and is sensitive to various environmental conditions.

The present teachings provide a filamentous fungus comprising two or more polynucleotides that encode two or more heterologous polypeptides and a polynucleotide encoding a homologous polypeptide. The filamentous fungus is capable of expressing the heterologous and homologous polypeptides that form a functional mixture. In some embodiments, the filamentous fungus contains a first polynucleotide and a second polynucleotide, encoding a first heterologous polypeptide and a second heterologous polypeptide, respectively, and a third polynucleotide encoding a homologous polypeptide. In some embodiments, the filamentous fungus contains an additional polynucleotide, a fourth polynucleotide, encoding a third heterologous polypeptide. In some embodiments, the filamentous fungus contains four or more polynucleotides encoding four or more heterologous polypeptides and one or more polynucleotides encoding one or more homologous polypeptides.

According to the present teachings, a functional mixture includes any mixture of polypeptides, provided that such mixture has at least one function, biological or otherwise, that is derived from at least two or three polypeptides from the mixture. In other words, at least two or three polypeptides from the mixture contribute, at a detectable level, to the function of the polypeptide mixture. In some embodiments, the functional mixture includes at least three polypeptides and has a function derived from at least two or three of the polypeptides from the mixture. In some other embodiments, the functional mixture includes at least three polypeptides and has an enzymatic function derived from at least two or three polypeptides from the mixture. In some embodiments, the functional mixture includes at least three polypeptides and has a cellulase function derived from at least two or three of the polypeptides of the mixture. In some embodiments, the functional mixture includes four polypeptides and has a function derived from two, three or four of the polypeptides from the mixture.

In some embodiments, the functional mixture includes a function that corresponds to or is an improvement of any activity, e.g., secretable protein activity including without any limitation, cellulase activity, saccharification activity or thermal stability associated with or provided by a filamentous fungus. In some embodiments, the functional mixture includes a function derived from the activity of exo-cellobiohydrolases, endoglucanases, or beta-glucosidases or any combination thereof. In some embodiments, the functional mixture does not include any bacterial enzyme in combination with its carrier filamentous protein. In some embodiments, the functional mixture does not form any antibody or functional antibody fragments, e.g., Fab, single chain antibody, etc.

In some embodiments, the polynucleotides encoding heterologous or homologous polypeptides are operably linked to one or more promoters. The promoter can be any suitable promoter now known, or later discovered, in the art. In some embodiments, the polynucleotides are expressed under a promoter native to the filamentous fungus. In some embodiments, the polynucleotides are under a heterologous promoter. In some embodiments, the polynucleotides are expressed under a constitutive or inducible promoter. Examples of promoters that can be used include, but are not limited to, a cellulase promoter, a xylanase promoter, the 1818 promoter (previously identified as a highly expressed protein by EST mapping Trichoderma). In some embodiments, the promoter is a cellulase promoter of the filamentous fungus. In some embodiments, the promoter is an exo-cellobiohydrolase, endoglucanase, or beta-glucosidase promoter. In some embodiments, the promoter is a cellobiohydrolase I (cbh 1) promoter. Non-limiting examples of promoters include a cbh1, cbh2, egl1, egl2, egl3, egl4, egl5, pki1, gpd1, xyn1, and xyn2 promoter. Further, two or more of the polynucleotides encoding the heterologous or homologous polypeptides, or portions thereof, can be fused together to form a fusion polynucleotide. The fusion polynucleotide can be operably linked to any suitable promoter as discussed above.

In some embodiments, the first polynucleotide encoding a first heterologous polypeptide is operably linked to a first promoter. The first promoter can, but need not, be different from the promoter or promoters to which the second or third polynucleotides are operably linked. In some embodiments, the first polynucleotide is operably linked to a promoter of a gene encoding the homologous polypeptide.

In some embodiments, a polynucleotide, e.g., the second polynucleotide, encoding a second heterologous polypeptide, is fused to another polynucleotide, e.g., with the third polynucleotide encoding a homologous polypeptide, to form a fusion polynucleotide. The fusion polynucleotide can be operably linked to any suitable promoter, including, but not limited to, a promoter of a gene encoding the homologous polypeptide. The fusion polynucleotide encodes a fusion polypeptide or fusion protein that comprises two polypeptides, or domains or portions thereof. The portions or domains of the polypeptides can be any portion or domain of the polypeptides that either has at least one function, biological or otherwise, or becomes functional when combined into a fusion polypeptide or when combined with the other polypeptides of the functional mixture. In some embodiments, the fusion protein comprises the second heterologous polypeptide and the homologous polypeptide.

In some embodiments, the fusion polynucleotide encodes a fusion protein that comprises two polypeptides, e.g., the second heterologous polypeptide and the homologous polypeptide, separated by a linker or a linker region. The linker can be any suitable linker for connecting two polypeptides. The linker region generally forms an extended, semi-rigid spacer between independently folded peptide domains. A linker region between the polypeptides of the fusion protein may be beneficial in allowing the polypeptides to fold independently. In some embodiments, the linker is from glucoamylase from Aspergillus species and CBHI linkers from Tricoderma species. In some embodiments, the linker can, but need not, be a portion of the polypeptides comprising the fusion protein. In some embodiments, the polypeptides of the fusion protein are second heterologous polypeptide and the homologous polypeptide.

In some embodiments, the fusion polynucleotide encodes a fusion protein that comprises two polypeptides separated by a linker or linker region and a cleavage site. In some embodiments, the polypeptides of the fusion protein are the second heterologous polypeptide and the homologous polypeptide. In general, the cleavage site will be located within the linker region and will allow the separation of the sequences bordering the cleavage site. The cleavage site can comprise any sequence that can be cleaved by any means now known or later developed, including, but are limited to, cleavage by a protease or after exposure to certain chemicals. Examples of such sequences include, but are not limited to, a kexin cleavage site, e.g., a KEX2 recognition site which includes codons for the amino acids Lys Arg, trypsin protease recognition sites of Lys and Arg, and the cleavage recognition site for endoproteinase-Lys-C.

In some embodiments, the filamentous fungus of the present teachings further comprises a polynucleotide encoding a selectable marker. The marker can be any suitable marker that allows the selection of transformed host cells. In general, a selectable marker will be a gene capable of expression in host cell which allows for ease of selection of those hosts containing the vector. As used herein, the term generally refers to genes that provide an indication that a host cell has taken up an incoming DNA of interest or some other reaction has occurred. Generally, selectable markers are genes that confer antimicrobial resistance or a metabolic advantage on the host cell to allow cells containing the exogenous DNA to be distinguished from cells that have not received any exogenous sequence during the transformation. Examples of such selectable markers include but are not limited to antimicrobials, (e.g., kanamycin, erythromycin, actinomycin, chloramphenicol and tetracycline). Additional examples of markers include, but are not limited to, a T. reesei pyr4, acetolactate synthase, Streptomyces hyg, Aspergillus nidulans amdS gene and an Aspergillus niger pyrG gene.

In some embodiments, the filamentous fungus of the present teachings further comprises, and is capable of expressing, a fourth polynucleotide encoding a third heterologous polypeptide. The heterologous or homologous polypeptides can be naturally occurring polypeptides or variants thereof. In some embodiments, one or more of the heterologous polypeptides may be variants of the homologous polypeptides. For example, the first heterologous polypeptide can be a modified homologous polypeptide. In some embodiments, the first and second heterologous polypeptides are modified homologous polypeptides. In some embodiments, the first and second heterologous polypeptides are modified homologous polypeptides and the filamentous fungus contains a fourth polynucleotide encoding a third heterologous polypeptide. The third heterologous may, or may not be a modified homologous polypeptide.

The heterologous and homologous polypeptides of the present teachings can be any desired polypeptide that, when mixed with the other polypeptides of the present teachings produces a functional mixture that has at least one function, biological or otherwise, that is derived from at least two or three polypeptides from the mixture. In some embodiments, the mixture of the heterologous and homologous polypeptides allow the functional mixture to display improved function with respect to an activity of, associated with, or provided by a filamentous fungus. In some embodiments, the activities include, but are not limited to, an improved secretable protein activity, improved saccharification activity or thermal stability, i.e., stability at higher temperatures, or altered pH values and/or sustained activity for greater time periods at the same temperature.

In some embodiments, the heterologous or homologous polypeptides do not include any bacterial enzyme in combination with its carrier filamentous protein. In some embodiments, the heterologous or homologous polypeptides do not combine to form any antibody or functional antibody fragments, e.g., Fab, single chain antibody, etc.

In some embodiments, one or more of the first or the second heterologous polypeptide or the homologous polypeptide is an enzyme or a portion thereof. In some embodiments, the first or the second heterologous polypeptide or the homologous polypeptide is a cellulase, hemicellulase, xylanase, mannanase or a domain or portion thereof. In some embodiments, the first or the second heterologous polypeptide or the homologous polypeptide is a cellulase or a portion thereof. In some embodiments, the first and the second heterologous polypeptides and the homologous polypeptide combine to form a functional mixture of cellulases.

In some embodiments, the first or second heterologous polypeptide or the homologous polypeptide is a cellulase selected from the group of: exo-cellobiohydrolases, endoglucanases, beta-glucosidases or portions thereof. The first or the second heterologous polypeptide, the homologous polypeptide and, if present, the third heterologous polypeptide, can be selected from the group of: exo-cellobiohydrolases, endoglucanases, beta-glucosidases or domains thereof without any restriction. In some embodiments, more than one polypeptide, heterologous or homologous, can belong to the same class or group of cellulases. For example, two or more of the polypeptides can belong to the class of exo-cellobiohydrolases. In some embodiments, one of the heterologous polypeptide belongs to the same class of cellulases as the homologous polypeptide. In some embodiments, the heterologous and homologous polypeptides are the same member of the class, but have sequences from different origins.

In some embodiments, the filamentous fungus of the present teachings contains a first polynucleotide and a second polynucleotide, encoding a first heterologous polypeptide and a second heterologous polypeptide, respectively, wherein the first heterologous polypeptide is an exo-cellobiohydrolase and the second heterologous polypeptide is an endoglucanase. In some embodiments, the first heterologous polypeptide is an exo-cellobiohydrolase, classified as EC 3.2.1.91, and the second heterologous polypeptide is an endoglucanase, classified as EC 3.2.1.4. In some embodiments, the first heterologous polypeptide is an exo-cellobiohydrolase selected from the group consisting of GH family 5, 6, 7, 9, 48, and wherein the second heterologous polypeptide is an endoglucanase selected from the group consisting of GH family 5, 6, 7, 8, 9, 12, 17, 31, 44, 45, 48, 51, 61, 64, 74 and 81.

As discussed above the heterologous and homologous polypeptides of the present teachings can be selected without restriction from the classes of cellulase enzymes. Exemplary combinations of enzymes are provided herein. In some embodiments, the first heterologous polypeptide is an exo-cellobiohydrolase, the second heterologous polypeptide is an endoglucanase, and the homologous polypeptide is an exo-cellobiohydrolase. In some embodiments, the first heterologous polypeptide is a first exo-cellobiohydrolase, the second heterologous polypeptide is an endoglucanase, the homologous polypeptide is a second exo-cellobiohydrolase, and the first exo-cellobiohydrolase and the second exo-cellobiohydrolase correspond to the same member of cellobiohydrolases, for example, both the first and second exo-cellobiohydrolases are CBHI or both are CBHII.

The filamentous fungi of the present teachings can be any filamentous fungus recognized by those of skill in the art. In some embodiments, the filamentous fungi include, but are not limited to the following genera: Aspergillus, Acremonium, Aureobasidium, Beauveria, Cephalosporium, Ceriporiopsis, Chaetomium paecilomyces, Chrysosporium, Claviceps, Cochiobolus, Cryptococcus, Cyathus, Endothia, Endothia mucor, Fusarium, Gilocladium, Humicola, Magnaporthe, Myceliophthora, Myrothecium, Mucor, Neurospora, Phanerochaete, Podospora, Paecilomyces, Penicillium, Pyricularia, Rhizomucor, Rhizopus, Schizophylum, Stagonospora, Talaromyces, Trichoderma, Thermomyces, Thermoascus, Thielavia, Tolypocladium, Trichophyton, and Trametes pleurotus. In some embodiments, the filamentous fungi include, but are not limited to the following: A. nidulans, A. niger, A. awomari, e.g., NRRL 3112, ATCC 22342 (NRRL 3112), ATCC 44733, ATCC 14331 and strain UVK 143f, A. oryzae, e.g., ATCC 11490, N. crassa, Trichoderma reesei, e.g., NRRL 15709, ATCC 13631, 56764, 56765, 56466, 56767, and Trichoderma viride, e.g., ATCC 32098 and 32086.

In some embodiments, the filamentous fungus of the present teachings is Trichoderma. In some embodiments, the filamentous fungus of the present teachings is Trichoderma reesei. In some embodiments, the heterologous polypeptides can be from any of the following: Humicola grisea, Acidothermus cellulolyticus, Thermobifida fusca, or Penicillium funiculosum. In some embodiments, the heterologous polypeptides is from Humicola grisea, Acidothermus cellulolyticus, Thermobifida, e.g. Thermobifida fusca, or Penicillium funiculosum and the homologous polypeptide is from Trichoderma reesei.

Exemplary combinations of heterologous and homologous polypeptides are provided herein. In some embodiments, the heterologous and the homologous polypeptides of the functional mixture can be selected from the group consisting of T. reesei EGI, EGII, EGIII (CEL7B, 5A, 12A, respectively), variants of CEL12A, H. grisea EGIII, T. fusca E5 and E3 and A. cellulolyticus E1 and GH74. In some embodiments, the heterologous polypeptides of the functional mixture can be exo-endo cellulase fusion construct. In some embodiments, the fusion protein has cellulolytic activity comprising a catalytic domain derived from a fungal exo-cellobiohydrolase and a catalytic domain derived from an endoglucanase. Suitable, but non-limiting examples are provided in U.S. Patent Application Publication No. 20060057672.

In some embodiments, the heterologous polypeptides of the functional mixture can be variants of H. jecorina CBH I, a Cel7 enzyme. In some embodiments the cellobiohydrolases can be have improved thermostability and reversibility, including but not limited to those described in U.S Patent Application Publication No. 20050277172 and 20050054039.

In some embodiments, the heterologous polypeptides of the functional mixture can be variants of H. jecorina CBH 2, a Cel7 enzyme. In some embodiments the cellobiohydrolases can be have improved thermostability and reversibility, including but not limited to those described in U.S Patent Application Publication No. 20060205042.

In some embodiments, the host filamentous fungus is T. reesei, the first heterologous polypeptide is Humicola grisea CBHI, the second heterologous polypeptide is Acidothermus cellulolyticus endoglucanase 1, and the homologous polypeptide is Trichoderma reesei CBHI. In some embodiments, the filamentous fungus is T. reesei and the first heterologous polypeptide or the second heterologous polypeptide is selected from the group consisting of Penicillium funiculosum cellobiohydrolase CBHI, Thermobifida endoglucanases E3, Thermobifida endoglucanases E5, Acidothermus cellulolyticus GH74-core and GH48.

In some embodiments, the filamentous fungus comprises a fourth polynucleotide encoding a third heterologous polypeptide. Here, the first polypeptide is a modified T. reesei CBHI, the second heterologous polypeptide is a modified T. reesei CBHII, the third heterologous polypeptide is Acidothermus cellulolyticus endoglucanase 1, and the homologous polypeptide is T. reesei CBHI.

The present teachings also provides for functional mixtures with improved properties and/or activities. In some embodiments, the first heterologous polypeptide is an exo-cellobiohydrolase, the second heterologous polypeptide is an endoglucanase, and the homologous polypeptide is an exo-cellobiohydrolase. Here, the first heterologous polypeptide, the second heterologous polypeptide and the homologous polypeptide form a mixture of thermostable cellulases.

Further, in some embodiments, the present teachings provide that the polynucleotides encoding the heterologous as well as the homologous polypeptides can be extrachromosomal, i.e., in a vector or plasmid or alternatively, the polynucleotides can be integrated within the chromosomes of filamentous fungus host. In some embodiments, the filamentous fungus host has at least one polynucleotide encoding the first, second or third heterologous polypeptide or the homologous polypeptide integrated into its genome. In some embodiments, the filamentous fungus host has at least one polynucleotide encoding the first, second or third heterologous polypeptide or the homologous polypeptide integrated into its genome and at least one polynucleotide encoding a heterologous or homologous polypeptide in a stable vector transformed into the host.

In some embodiments, the host is T. reesei with at least one polynucleotide encoding the first or second heterologous polypeptide or the homologous polypeptide integrated into its genome. In some embodiments, the host is T. reesei with two polynucleotides integrated into its genome. The polynucleotides encode either the first, second, or, if present, the third heterologous polypeptide or the homologous polypeptide. In some embodiments, one or more polynucleotides expressing either a heterologous or homologous exo-cellobiohydrolase are integrated into the genome of a T. reesei host. In some embodiments, a polynucleotide encoding a heterologous endoglucanase is integrated into the genome of a T. reesei host. In some embodiments, a polynucleotide encoding a heterologous endoglucanase and a polynucleotide encoding either a heterologous or homologous exo-cellobiohydrolase are integrated into the genome of a T. reesei host. It is understood that when only one or two of the three or four polynucleotides that encode the polypeptides of the functional mixture are integrated into the host genome, the remaining polynucleotides are transformed into the host and are present in a vector or plasmid. In some embodiments, the filamentous fungus contains a first polynucleotide and a second polynucleotide, encoding a first heterologous polypeptide and a second heterologous polypeptide, respectively, and a third polynucleotide encoding a homologous polypeptide and all three polynucleotides are extrachromosomal.

The present teachings also provide a culture medium comprising a population of the filamentous fungi described above. The culture medium can be solid, semi-solid or liquid and suitably chosen depending on the host as well as the polypeptides expressed therein.

Further, the present teachings also provide a polypeptide mixture comprising the first heterologous polypeptide, the second heterologous polypeptide, and the homologous polypeptide obtained from the filamentous fungi described herein. In some embodiments, the polypeptide mixture is a mixture of enzymes or domains thereof. In some embodiments, the polypeptide mixture is a mixture of cellulases, hemicellualses, xylanases, mannanases or domains thereof.

In addition, the present teachings provide a method of producing a mixture of polypeptides comprising obtaining a polypeptide mixture from the filamentous fungi described herein. The polypeptide mixture contains a first heterologous polypeptide, a second heterologous polypeptide, and a homologous polypeptide. In some embodiments, the mixture of polypeptides contains a third heterologous polypeptide. As discussed above, the mixture of polypeptides is a functional mixture. In some embodiments, the mixture of polypeptides is a mixture of enzymes or domains thereof. In some embodiments, the mixture of polypeptides is a mixture of cellulases, hemicellualses, xylanases, mannanases or domains thereof.

In some embodiments, the mixture of polypeptides is a mixture of cellulases comprising a first heterologous polypeptide that is an exo-cellobiohydrolase, a second heterologous polypeptide that is an endoglucanase, and a homologous polypeptide that is an exo-cellobiohydrolase. In some embodiments, the mixture of cellulases contains a first heterologous polypeptide that is a first exo-cellobiohydrolase, a second heterologous polypeptide that is an endoglucanase, and a homologous polypeptide that is a second exo-cellobiohydrolase. Here, the first exo-cellobiohydrolase and the second exo-cellobiohydrolase correspond to the same member of cellobiohydrolases. In some embodiments, the first and second exo-cellobiohydrolase are CBHI. In some embodiments, the first and second exo-cellobiohydrolase are CBHII.

As will be apparent to one of skill in the art, several other combinations of heterologous and homologous polypeptides can be expressed in the filamentous fungi of the present teachings. Another exemplary mixture of cellulases comprises a first heterologous polypeptide that is Humicola grisea CBHI, a second heterologous polypeptide that is Acidothermus cellulolyticus endoglucanase 1, and a homologous polypeptide that is Trichoderma reesei CBHI.

Aspects of the present teachings may be further understood in light of the following examples, which should not be construed as limiting the scope of the present teachings. It will be apparent to those skilled in the art that many modifications, both to materials and methods, may be practiced without departing from the present teachings.

### EXAMPLES

### 7.1 Example 1 Construction of the Tripartite Strain

The Tripartite strain consists of the following three parts: (i) a T. reesei cellulase production strain; (ii) nucleic acid comprising a Humicola grisea cbh1 gene in that strain; and (iii) an exo-endo cellulase fusion of T. reesei cbh1 with Acidothermus cellulolyticus endoglucanasel.

Construction of a CBH1-E1 Fusion Vector

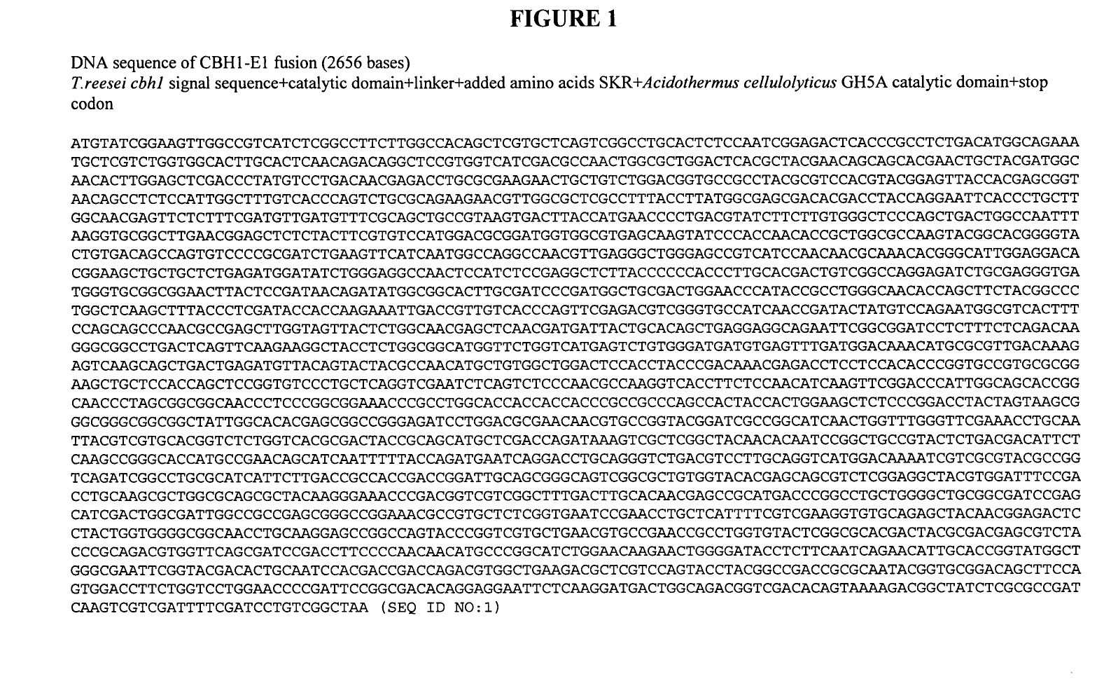

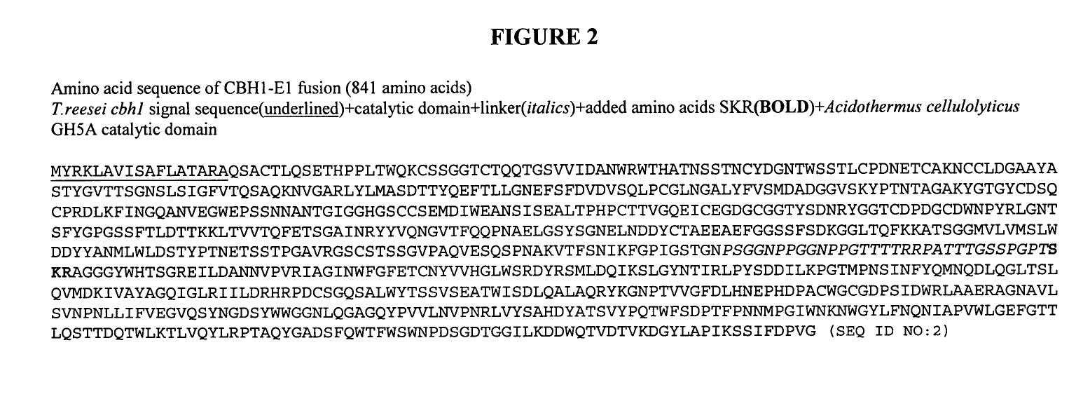

The CBH 1-E1 fusion construct included the T. reesei cbhI promoter; the T. reesei cbhI gene sequence from the start codon to the end of the cbhI linker and an additional 12 bases of DNA 5′ to the start of the endoglucanase coding sequence, the endoglucanase coding sequence, a stop codon and the T. reesei cbhI terminator. The nucleotide sequence (SEQ ID NO: 1) of the heterologous cellulase fusion construct comprised 2656 bases (see FIG. 1), and included the T. reesei cbhI signal sequence; the catalytic domain of the T. reesei cbhI; the T. reesei cbhI linker sequence; a kexin cleavage site which includes codons for the amino acids SKR and the sequence coding for the Acidothermus cellulolyticus GH5A-E1 catalytic domain. The predicted amino acid sequence (SEQ ID NO: 2) of the cellulase fusion protein based on the nucleic acid sequence of FIG. 1 is shown in FIG. 2. The additional 12 DNA bases, ACTAGTAAGCGG (nucleotides 1565 to 1576 of SEQ ID NO: 1) code for the restriction endonuclease SpeI and the amino acids Thr, Ser, Lys, and Arg.

The plasmid E1-pUC19 which contained the open reading frame for the E1 gene locus was used as the DNA template in a PCR reaction. (Equivalent plasmids are described in U.S. Pat. No. 5,536,655, which also describes the cloning of the E1 gene from the actinomycete Acidothermus cellulolyticus ATCC 43068, Mohagheghi A. et al., 1986). Standard procedures for working with plasmid DNA and amplification of DNA using the polymerase chain reaction (PCR) are found in Sambrook, et al., 2001.

The following two primers were used to amplify the coding region of the catalytic domain of the E1 endoglucanase.

Forward Primer 1 = EL-316 (containg a SpeI site):
(SEQ ID NO: 3)
GCTTATACTAGTAAGCGCGCGGGCGGCGGCTATTGGCACAC;
Reverse Primer 2 = EL-317 (containing an AscI
site and stop codon-reverse compliment):
(SEQ ID NO: 4)
GCTTATGGCGCGCCTTAGACAGGATCGAAAATCGACGAC.

The reaction conditions were as follows using materials from the PLATINUM Pfx DNA Polymerase kit (Invitrogen, Carlsbad, Calif.): 1 μl dNTP Master Mix (final concentration 0.2 mM); 1 μl primer 1 (final conc 0.5 μM); 1 μl primer 2 (final conc 0.5 μM); 2 μl DNA template (final conc 50-200 ng); 1 μA 50 mM MgSO₄ (final conc 1 mM); 5 μl 10×Pfx Amplification Buffer; 5 μl 10×PCRx Enhancer Solution; 1 μl Platinum Pfx DNA Polymerase (2.5 U total); 33 μl water for 50 μl total reaction volume.

Amplification parameters were: step 1: 94° C. for 2 min (1st cycle only to denature antibody bound polymerase); step 2: 94° C. for 45 sec; step 3: 60° C. for 30 sec; step 4: 68° C. for 2 min; step 5: repeated step 2 for 24 cycles; and step 6: 68° C. for 4 min.

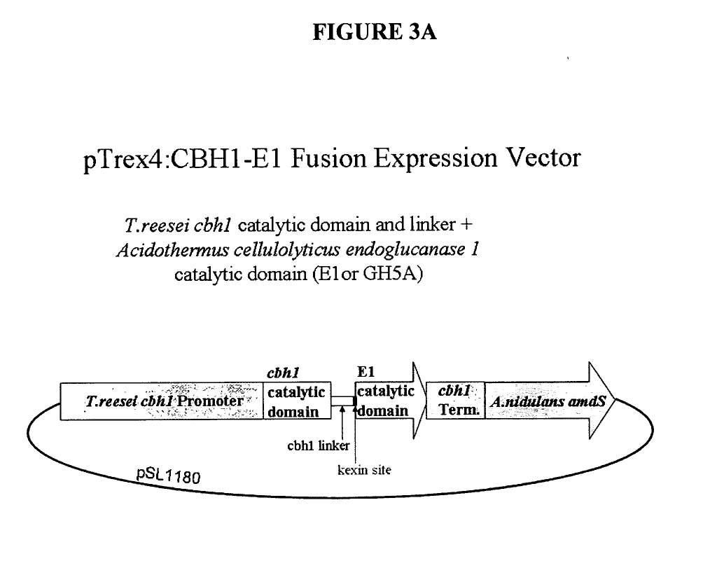

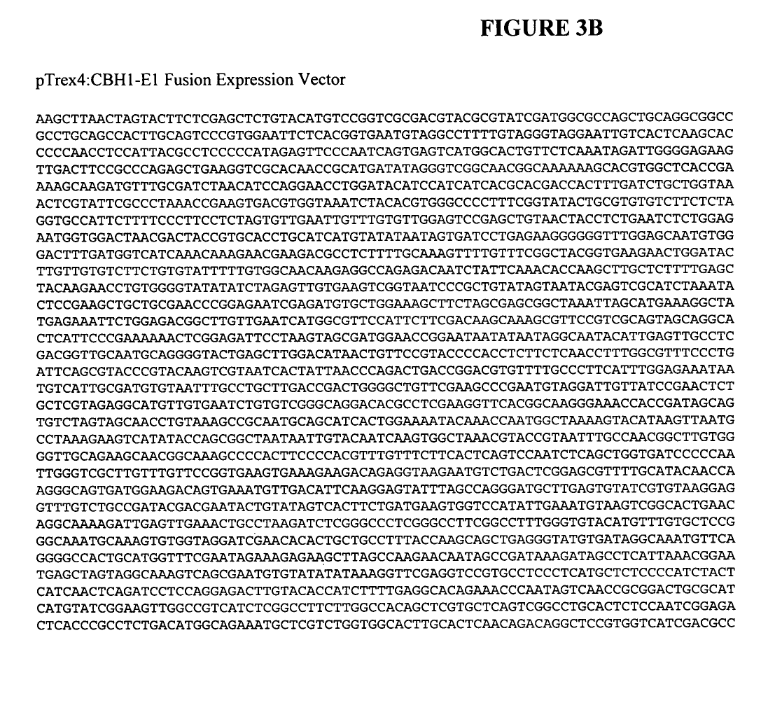

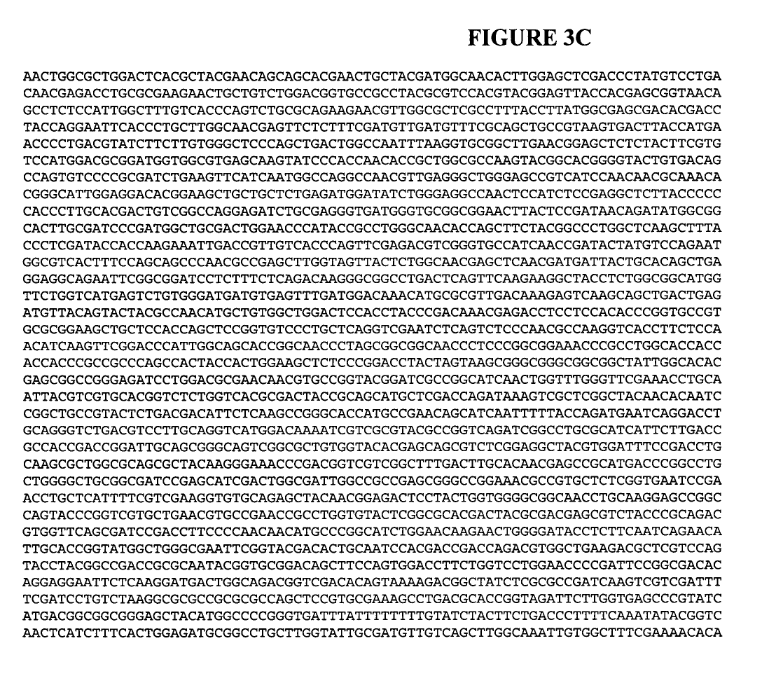

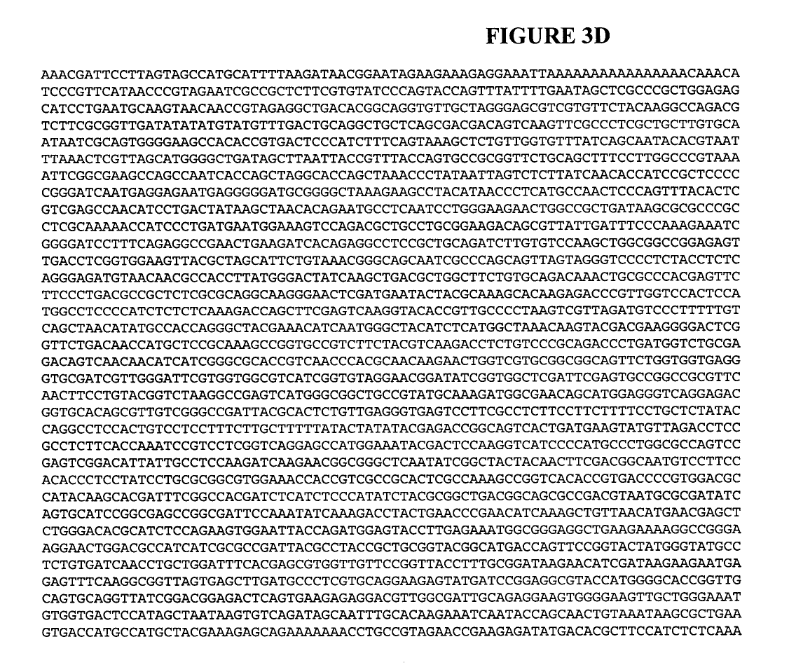

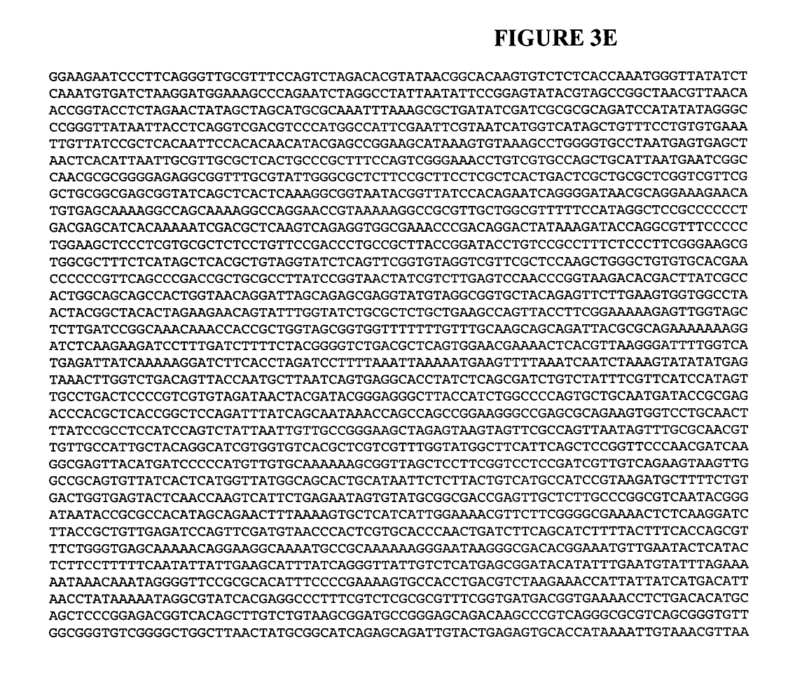

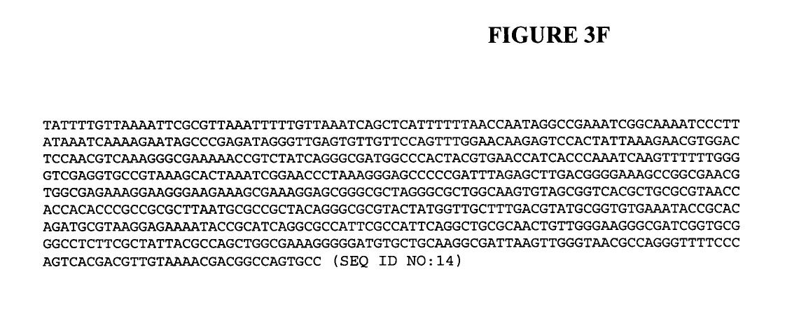

The appropriately sized PCR product was cloned into the Zero Blunt TOPO vector and transformed into chemically competent Top10 E. coli cells (Invitrogen Corp., Carlsbad, Calif.) plated onto to appropriate selection media (LA with 50 ppm kanamycin and grown overnight at 37° C. Several colonies were picked from the plate media and grown overnight in 5 ml cultures at 37° C. in selection media (LB with 50 ppm kanamycin) from which plasmid mini-preps were made. Plasmid DNA from several clones were restriction digested to confirm the correct size insert. The correct sequence was confirmed by DNA sequencing. Following sequence verification, the E1 catalytic domain was excised from the TOPO vector by digesting with the restriction enzymes SpeI and AscI. This fragment was ligated into the pTrex4 vector which had been digested with the restriction enzymes SpeI and AscI as shown in FIG. 3.

The ligation mixture was transformed into MM294 competent E. coli cells, plated onto appropriate selection media (LA with 50 ppm carbenicillin) and grown overnight at 37° C. Several colonies were picked from the plate media and grown overnight in 5 ml cultures at 37° C. in selection media (LB with 50 ppm carbenicillin) from which plasmid mini-preps were made. Correctly ligated CBH 1-E1 fusion protein vectors were confirmed by restriction digestion.

Construction of a H. grisea cbh1 Expression Vector

The H. grisea cbh1 expression construct included the T. reesei cbhI promoter; the H. grisea cbhI gene sequence, the T. reesei cbh1 terminator and the A. nidulans amdS selectable marker. These sequences can be assembled in a number of ways by those skilled in the art, one method is described as follows.

Genomic DNA was extracted from a sample of mycelia of Humicola grisea var. thermoidea (CBS 225.63). Genomic DNA may be isolated using any method known in the art. The following protocol may be used.

Cells were grown at 45° C. in 20 ml Potato Dextrose Broth (PDB) for 24 hours. The cells were diluted 1:20 in fresh PDB medium and grown overnight. Two milliliters of cells were centrifuged and the pellet washed in 1 ml KC (60 g KCl, 2 g citric acid per liter, pH adjusted to 6.2 with 1 M KOH). The cell pellet was resuspended in 900 μl KC. 100 μl (20 mg/ml) Novozyme was added, mixed gently and the protoplasting was followed microscopically at 37° C. until greater than 90% protoplasts were formed for a maximum of 2 hours. The cells were centrifuged at 1500 rpm (4600×G) for 10 minutes. 200 μl TES/SDS (10 mM Tris, 50 mM EDTA, 150 mM NaCl, 1% SDS) was added, mixed and incubated at room temperature for 5 minutes. DNA was isolated using a Qiagen mini-prep isolation kit (Qiagen). The column was eluted with 100 μl milli-Q water and the DNA collected.

An alternative method used the FastPrep method for isolating genomic DNA from H. grisea var thermoidea grown on PDA plates at 45° C. The system consists of the FastPrep Instrument as well as the FastPrep kit for nucleic acid isolation. (FastPrep is available from Qbiogene, MP Biomedicals United States, 29525 Fountain Pkwy., Solon, Ohio 44139).

Primers to PCR amplify the H. grisea cbh1 gene were based on NCBI ACCESSION D63515. They were designed to amplify from the H. grisea cbh1 coding start to the terminator. The sequence of the forward primer included the 4 nucleotides CACC to facilitate cloning into the vector TOPO pENTR to enable use of the Gateway cloning system (Invitrogen).

| Forward Primer: | |
|---|---|
| 5′ CACCATGCGTACCGCCAAGTTCGC 3′ | (SEQ ID NO: 5) |
| Reverse Primer: | |
| 5′ TTACAGGCACTGAGAGTACCAG 3′. | (SEQ ID NO: 6) |

PCR Reaction Conditions

The PCR product was cloned into pENTR/D, according to the Invitrogen Gateway system protocol. The vector was then transformed into chemically competent Top10 E. coli (Invitrogen) with kanamycin selection. Plasmid DNA from several clones was restriction digested to confirm the correct size insert, followed by sequencing to confirm the correct sequence. Plasmid DNA from one clone was added to a LR clonase reaction (Invitrogen Gateway system) with pTrex3g/amdS destination vector DNA.

Construction of pTrex3g

This section describes the construction of the basic vector used to express the genes of interest. The vector pTrex3g has been previously described, see for example, U.S. Patent Application Publication No. 20070015266. Briefly, the vector is based on the E coli vector pSL1180 (Pharmacia Inc., Piscataway, N.J., USA) which is a pUC118 phagemid based vector (Brosius, J. (1989) DNA 8: 759) with an extended multiple cloning site containing 64 hexamer restriction enzyme recognition sequences. It was engineered to become a Gateway destination vector (Hartley, J. L. et al., (2000) Genome Research 10: 1788-1795) to allow insertion using Gateway technology (Invitrogen) of any desired open reading frame between the promoter and terminator regions of the T. reesei cbh1 gene. The Aspergillus nidulans amdS gene was inserted for use as a selectable marker in transformation. A promoter and terminator were positioned to allow expression of a gene of interest.

The Details of pTrex3g are as Follows:

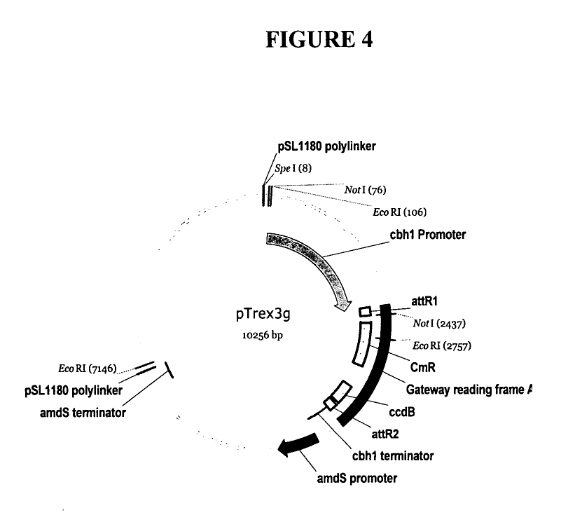

The vector is 10.3 kb in size. Inserted into the polylinker region of pSL1180 are the following segments of DNA: (i) a 2.2 by segment of DNA from the promoter region of the T. reesei cbh1 gene; (ii) the 1.7 kb Gateway reading frame A cassette acquired from Invitrogen that includes the attR1 and attR2 recombination sites at either end flanking the chloramphenicol resistance gene (CmR) and the ccdB gene; (iii) a 336 by segment of DNA from the terminator region of the T. reesei cbh1 gene; and (iv) a 2.7 kb fragment of DNA containing the Aspergillus nidulans amdS gene with its native promoter and terminator regions. FIG. 4 depicts the plasmid map of T. reesei expression vector pTrex3g.

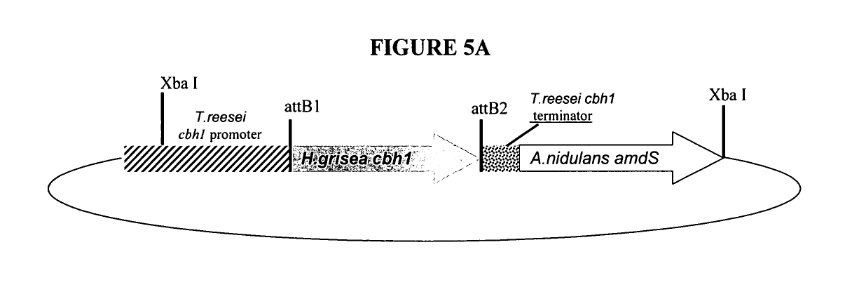

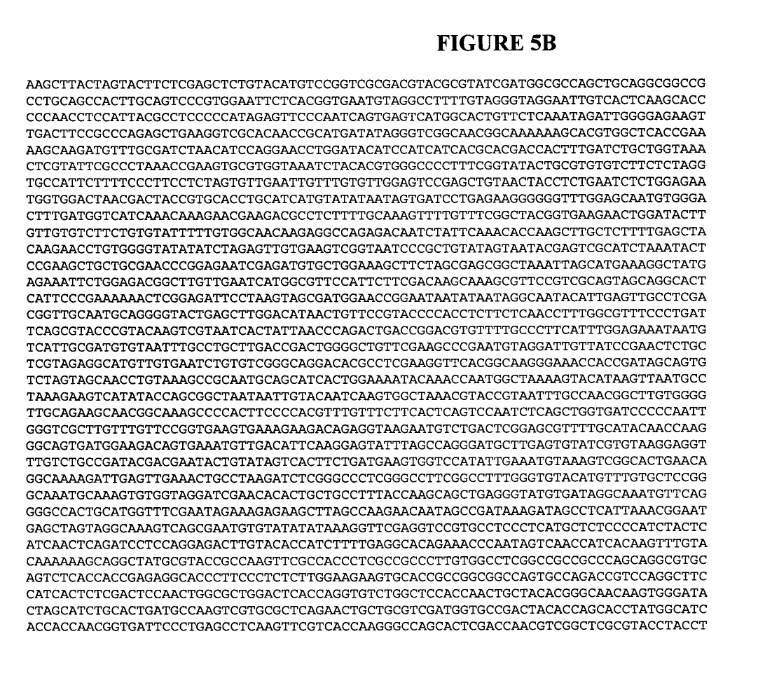

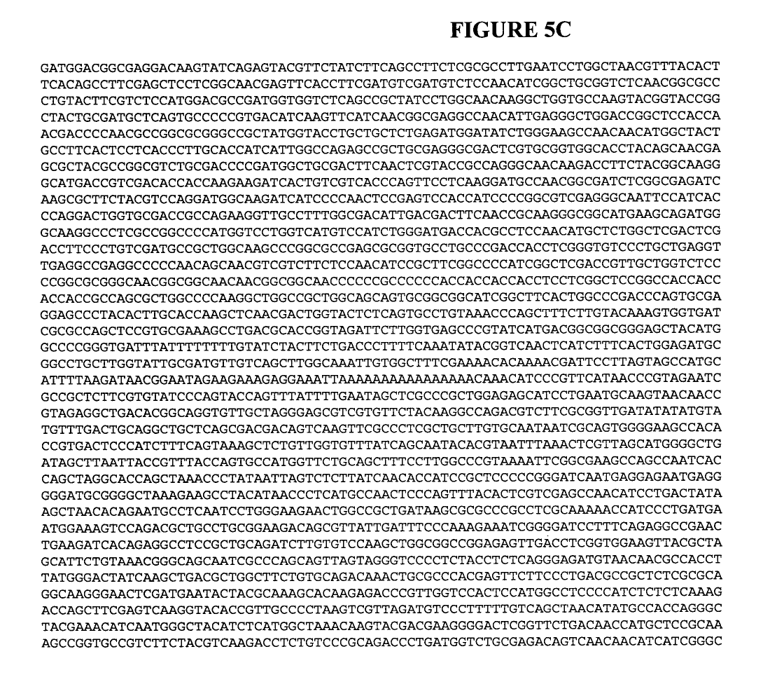

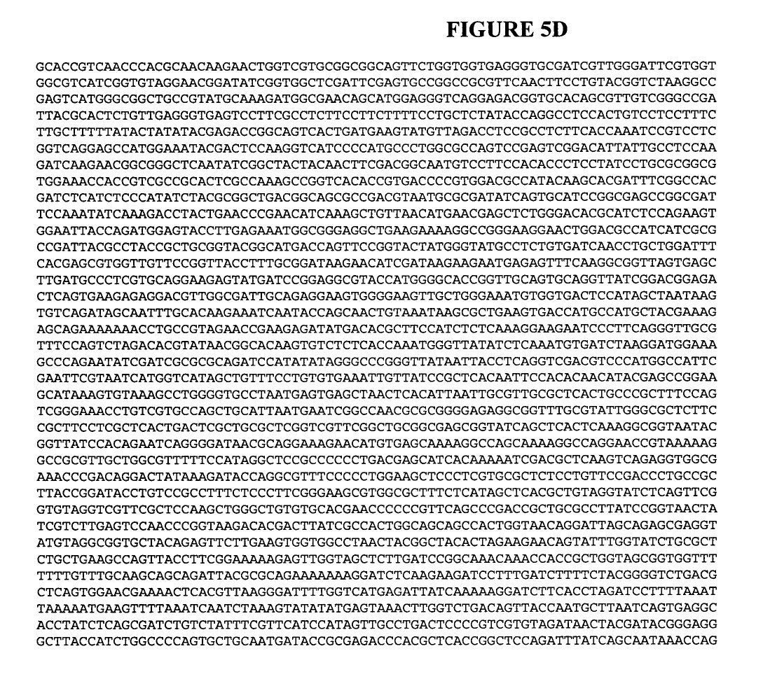

A clone of the H. grisea cbh1 in the vector pENTR, described above, was used to recombine with the pTrex3g-destination vector in a LR clonase reaction according to the manufactures instructions (Invitrogen). The H. grisea cbh1 replaced the CmR and ccdB genes of the pTrex3g destination vector with the H. grisea cbh1 from the pENTR/D vector. The recombination directionally inserted the H. grisea cbh1 between the T. reesei cbhI promoter and T. reesei cbh1 terminator of the destination vector. The recombination resulted in AttB sequences of 25 by flanking the H. grisea cbh1 both upstream and downstream. An aliquot of the LR clonase reaction was transformed into chemically competent Top10 E. coli cells (Invitrogen) and grown overnight with carbenicillin selection. Plasmid DNA, from several clones, were digested with appropriate restriction enzymes to confirm the correct insert size followed by sequencing to confirm the correct sequence. To provide DNA for transformation, plasmid DNA from a correct clone was digested with the endonuclease Xba1 to release the expression fragment including the T. reesei cbhI promoter: H. grisea cbh1: T. reesei cbhI terminator: A. nidulans amdS. This 6.2 kb fragment was isolated from the E. coli DNA by agarose gel extraction using standard techniques and transformed into a strain of T. reesei derived from the publicly available strain QM6a, as further described below. The expression vector including the two Xba I sites is shown schematically in FIG. 5A and the nucleotide sequence (SEQ ID NO: 7) of the expression vector is provided in FIG. 5B.

Co-Transformation and Fermentation of Trichoderma reesei

A derivative of T. reesei host strain RL-P37 (Sheir-Neiss, et al., 1984) which had undergone a number of mutagenensis steps to increase cellulase production, including deletion of the native cbh1 gene (Suominen, P. L. et al., 1993, Mol Gen Genet 241:523-30), was used as a host strain for transformations with the constructs of the present teachings.

Biolistic transformation of T. reesei with the H. grisea cbh1 expression construction and the fusion construct of T. reesei cbh1 and A. cellulolyticus E1 was performed using the protocol outlined below.

A suspension of spores (approximately 3.5×10⁸ spores/10 from a P-37 derived strain of T. reesei was prepared. Between 100 μl-200 μl of this spore suspension was spread onto the center of plates of MM acetamide medium. MM acetamide medium had the following composition: 0.6 g/L acetamide; 1.68 g/L CsCl; 20 g/L glucose; 20 g/L KH₂PO₄; 0.6 g/L CaCl₂.2H₂O; 1 ml/L 1000× trace elements solution; 20 g/L Noble agar; pH 5.5. 1000× trace elements solution contained 5.0 g/l FeSO₄.7H₂O, 1.6 g/l MnSO₄.H₂O, 1.4 g/l ZnSO₄.7H₂O and 1.0 g/l CoCl₂.6H₂O. The spore suspension was allowed to dry on the surface of the MM acetamide medium in a sterile hood.

Transformation of T. reesei was performed using a Biolistic® PDS-1000/He Particle Delivery System from Bio-Rad (Hercules, Calif.) following the manufacturer's instructions (Lorito, M. et al., 1993, Curr Genet 24:349-56). 60 mg of M10 tungsten particles were placed in a microcentrifuge tube. 1 mL of ethanol was added, the mixture was briefly vortexed and allowed to stand for 15 minutes. The particles were centrifuged at 15,000 rpm for 15 mins. The ethanol was removed and the particles were washed three times with sterile dH₂O before 1 mL of 50% (v/v) sterile glycerol was added. After ten seconds of vortexing to suspend the tungsten, 25 μl of tungsten/glycerol particle suspension was removed and placed into a microcentrifuge tube.

While continuously vortexing the 25 μl tungsten/glycerol particle suspension, the following were added in order, allowing 5′ incubations between additions; 2 μl (100-300 ng/μl) of H. grisea cbh1 expression vector (XbaI cut fragment), 2 μl (100-300 ng/μl) cbh1-E1 expression vector (XbaI cut fragment), 25 μl of 2.5M CaCl₂ and 10 μl of 0.1 M spermidine. After a 5′ incubation post spermidine addition, the particles were centrifuged for 3 seconds. The supernatant was removed; the particles were washed with 200 μl of 70% (v/v) ethanol and then centrifuged for 3 seconds. The supernatant was removed; the particles were washed with 200 μl of 100% ethanol and centrifuged for 3 seconds. The supernatant was removed and 24 μl 100% ethanol was added and mixed by pipetting. The tube was placed in an ultrasonic cleaning bath for approximately 15 seconds to further resuspend the particles in the ethanol. While the tube was in the ultrasonic bath, 8 μl aliquots of suspended particles were removed and placed onto the center of macrocarrier disks that were placed into a desiccator.

Once the tungsten/DNA solution had dried onto the macrocarrier (approximately 15′), it was placed into the bombardment chamber. Next a plate containing MM acetamide with spores and the bombardment process was performed using 1100 psi rupture discs according to the manufacturers instructions. After the bombardment of the plated spores with the tungsten/DNA particles, the plates were placed incubated at 28° C. Large transformed colonies were picked to fresh secondary plates of MM acetamide after 5 days (Penttila et al., (1987) Gene 61:155-164) and incubated another 3 days at 28° C. Colonies which showed dense, opaque growth on secondary plates were transferred to individual MM acetamide plates. These were grown another three days and transferred to potato dextrose agar plates (PDA) and allowed to incubate another 7-10 days at 28° C. to allow sporulation.

The expression of enzymes from the transformants was next evaluated in two stage shake flasks. They were first grown in an inoculum shake flask containing the following media: 22.5 g/L Proflo, 30 g/L a-Lactose.H₂O, 6.5 g/L (NH₄)₂SO₄, 2 g/L KH₂PO₄, 0.3 g/L MgSO₄.7H₂O, 0.26 g/L CaCL₂.2H₂O, 0.72 g/L CaCO₃, 2 ml of 10% Tween 80, 1 ml of 1000×TRI Trace Salts (1000×TRI Trace Salts consists of: 5 g/L FeSO₄.7H₂O, 1.6 g/L MnSO₄.H₂O, 1.4 g/L ZnSO₄.7H₂O). The conditions were as follows: 50 ml media in a 4 baffled, 250 ml shake flask (Bellco Biotechnology, 340 Edrudo Road, Vineland, N.J. 08360 USA), incubation at 28° C., shaking speed 225 RPM @ 2.5 cm diameter orbit). Transformants were inoculated into the inoculum shake flasks by transferring a 4 cm2 piece of PDA containing the transformant mycelia and spores.

After 2 days of growth in the inoculum flask, 5 ml was transferred into an expression shake flask containing 50 ml of the following media: 5 g/L (NH₄)₂SO₄, 33 g/L PIPPS Buffer, 9 g/L Bacto Casamino Acids, 4.5 g/L KH₂PO₄, 1.32 g/L CaCl₂.2H₂O, 1 g/L MgSO₄.7H₂O, 5 ml Mazu DF204 antifoam, 2.5 ml 400× T. reesei Trace Salts (400× T. reesei Trace Salts consists of: 175 g/L Citric Acid (anhydrous), 200 g/L FeSO₄.7H₂O, 16 g/L ZnSO₄.7H₂O, 3.2 g/L CuSO₄.5H₂O, 1.4 g/L MnSO₄.H₂O, 0.8 g/L H₃BO₃, added in order listed), pH is adjusted to 5.5, media is sterilized, post-sterilization, 40 ml of 40% lactose is added. Expression shake flask conditions were grown as follows: 4 baffled, 250 ml shake, incubation at 28° C., shaking speed 225. A sample was removed at 5 days, the supernate was analyzed on SDS-PAGE protein gels, coomassie stained.

### 7.2 Example 2 Four-Part Strain Construction

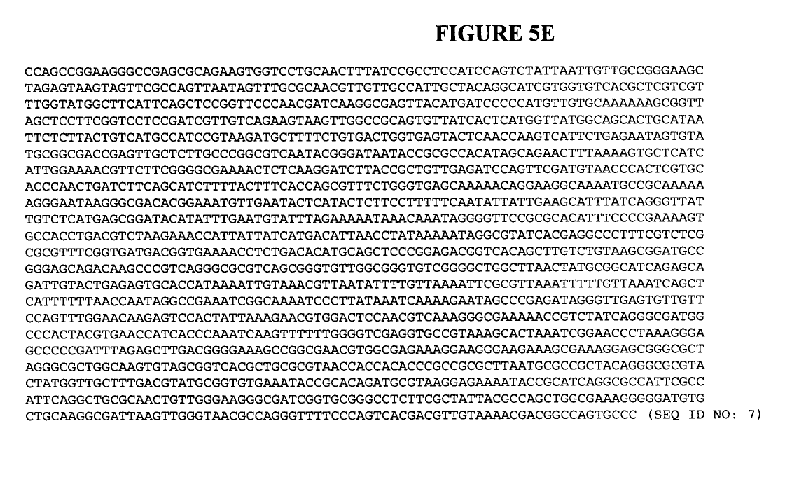

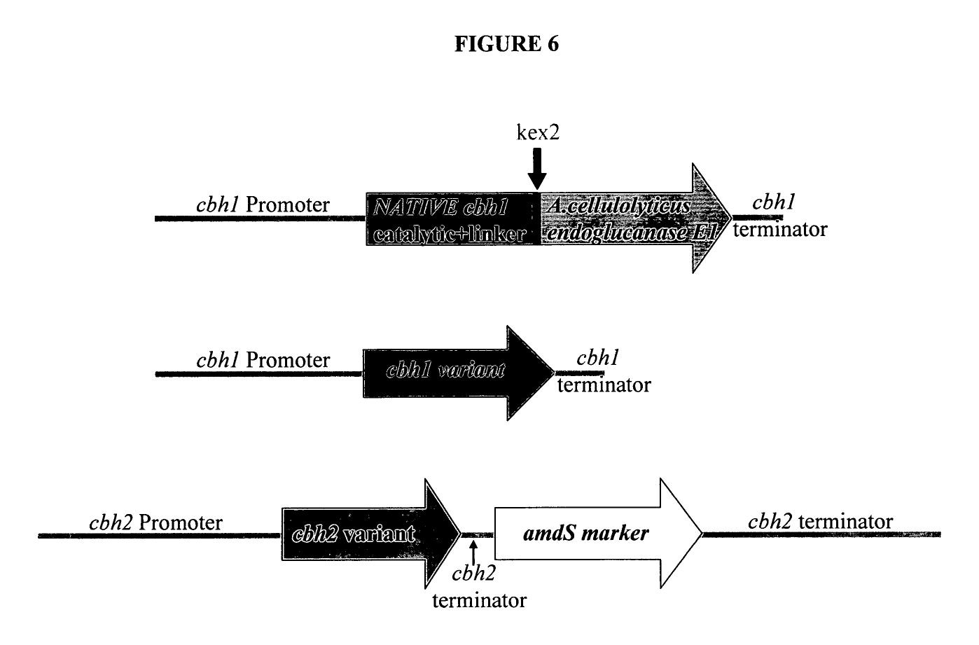

A strain was constructed which comprised four parts: (i) a host strain consisting of a cbhI deleted production strain; (ii) a nucleic acid sequence for expression of a cbhI-E1 fusion gene; (iii) a nucleic acid sequence for expression of a protein engineered thermostable T. reesei cbhI gene; and (iv) a nucleic acid sequence for expression of a protein engineered thermostable T. reesei cbhII gene. The DNA of all three expression fragments was co-transformed into the cbh1 deleted production strain as shown in FIG. 6.

T. reesei transformants were screened for the presence of all three expression fragments integrated into the genome. PCR primer pairs were designed to amplify each of the three expression fragments. 32 transformants that on the basis of PCR showed the presence of all three expression fragments were chosen for shake flask fermentation. Shake flasks were grown for three days, supernate samples were obtained and run in 8% tris-glycine NuPAGE (invitrogen) gels, 1 mm, in tris-glycine SDS running buffer. Sample preps were loaded at 20 μl/lane unless noted (8 μl supernate+2 μl reducing agent+10 μl of 2× tris-glycine SDS sample buffer) after incubating at 100° C. for 7 minutes followed by 5 minutes incubation on ice). Several of the 32 samples showed the high level presence of the expressed genes as evidenced by protein bands.

DNA encoding an amino acid sequence variant of the T. reesei cbhI and cbhII can be prepared by a variety of methods known in the art. These methods include, but are not limited to, gene synthesis, preparation by site-directed (or oligonucleotide-mediated) mutagenesis, PCR mutagenesis, and cassette mutagenesis of an earlier prepared DNA encoding the T. reesei cDNA sequence.

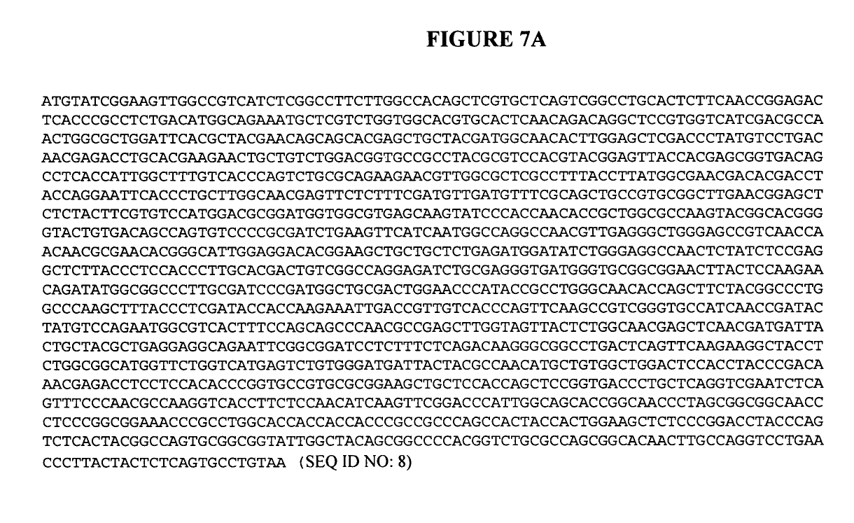

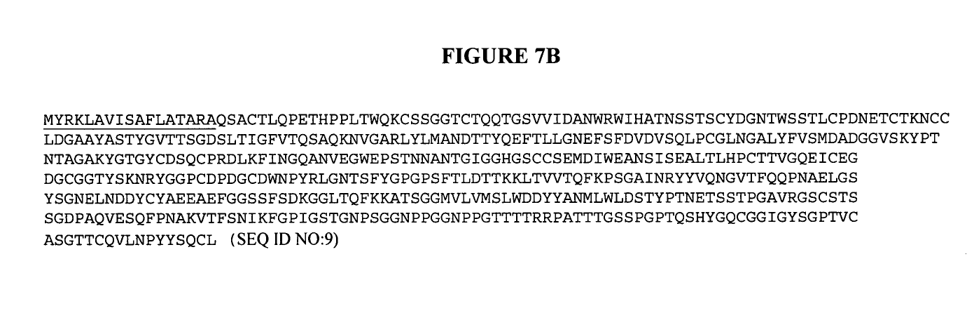

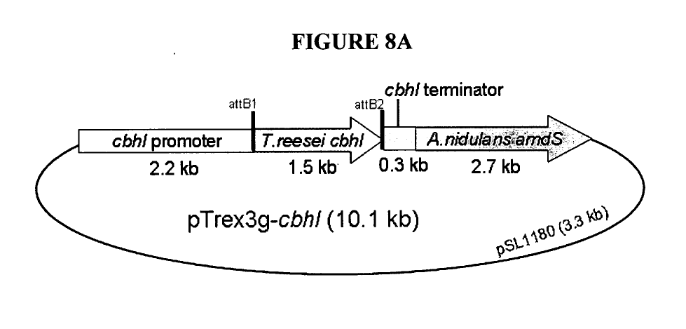

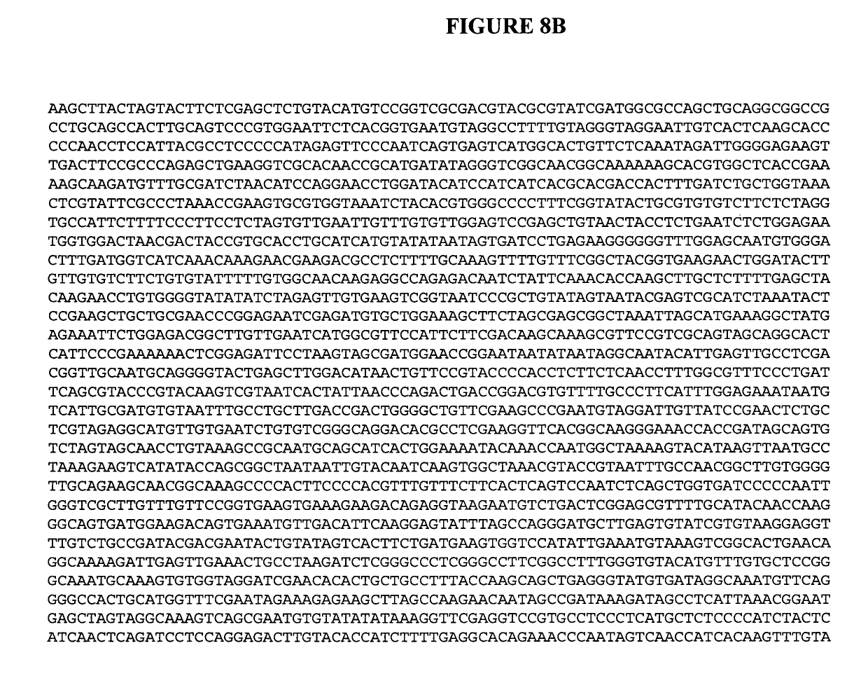

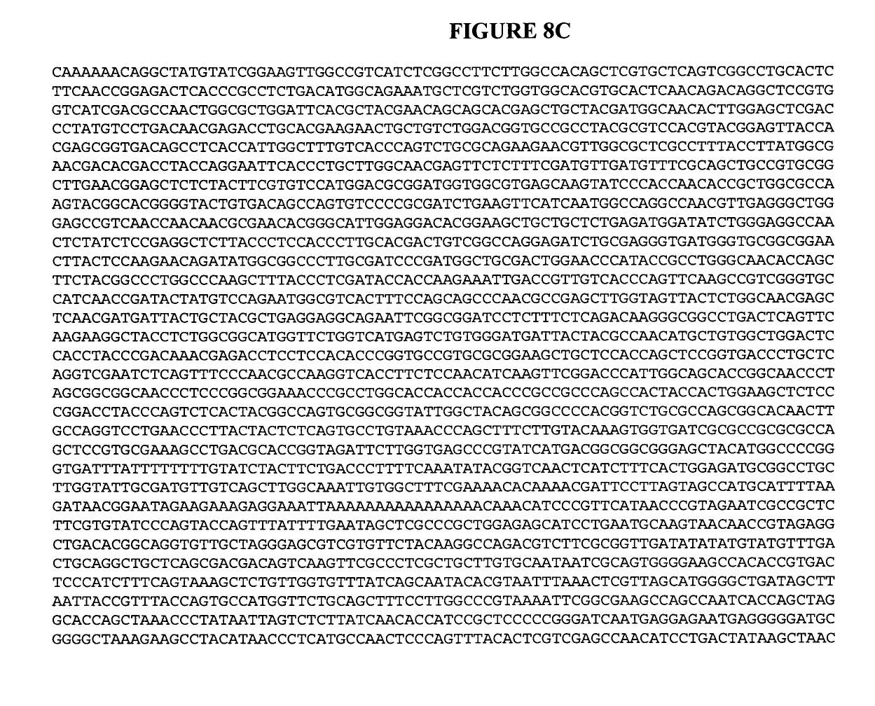

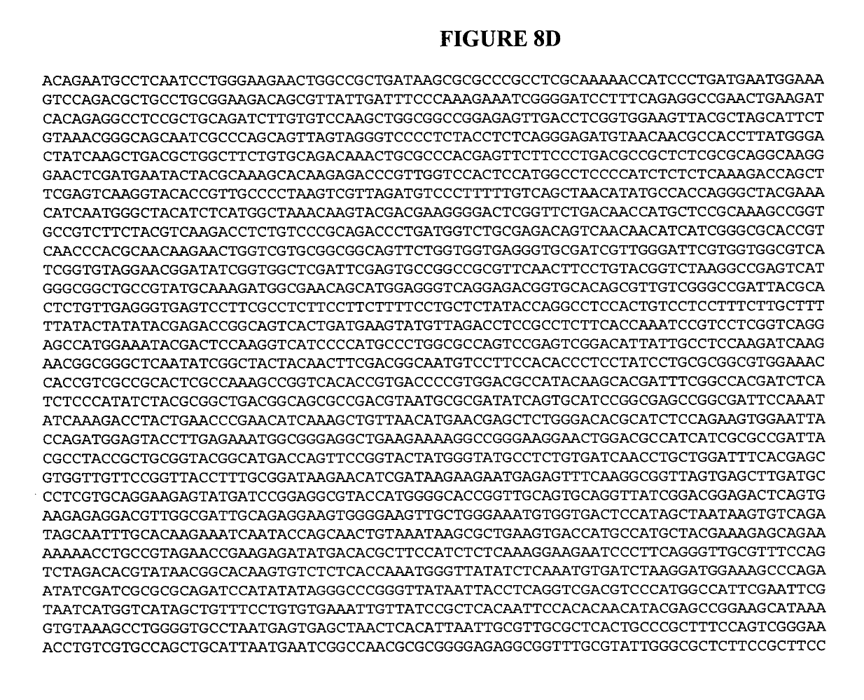

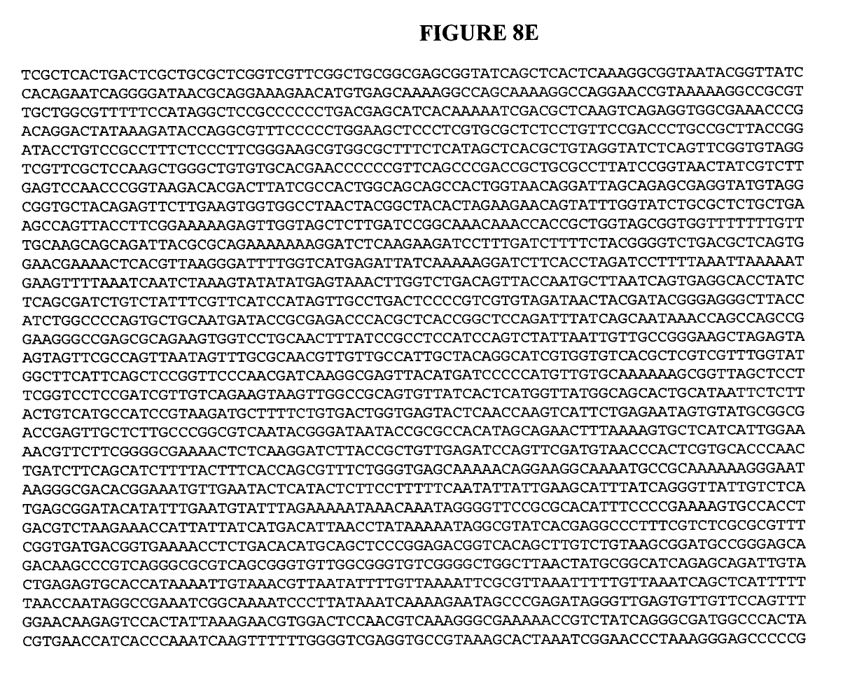

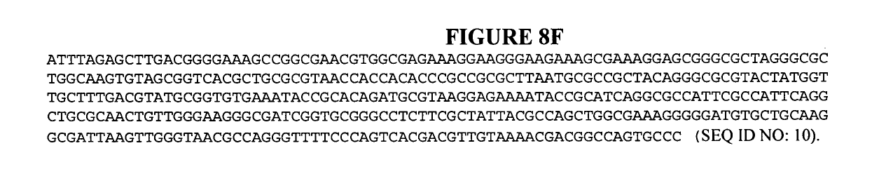

A vector was constructed in pTrex3g expressing an enzyme engineered T. reesei cbhI gene encoding an engineered protein with the following mutations in the mature amino acid sequence: S8P+T41I+N49S+A68T+N89D+S92T+S113N+S196T+P227L+D249K+T255P+S278P+E295K+T296P+T332Y+V403D+S411F. The DNA sequence from start to stop codon was 1545 bases (SEQ ID NO: 8) as provided in FIG. 7A. The sequence of the engineered CBHI protein (SEQ ID NO: 9) is provided in FIG. 7B (the CBHI signal sequence is underlined). A diagram of the cbhI expression vector pTrex3g-cbh1 is shown in FIG. 8A. The DNA sequence of the expression vector pTrex3g-cbh1 was 10145 bases (SEQ ID NO: 10) as provided in FIG. 8B.

A vector was constructed to express an enzyme engineered CBHII protein. The vector included the cbhII promoter, the engineered cbhII gene, the cbhII terminator, the A. nidulans acetamidase (amdS) as selectable marker, and additional flanking 3′ sequence to the cbhII terminator. The vector was constructed using the shuttle vector pCR-XL-TOPO (Invitrogen). The expression portion of the vector was excised from the shuttle vector by digestion of the plasmid with the unique restriction endonucleases NotI and SrfI, generating a fragment of approximately 10.68 kb in length which was used to transform T. reesei.

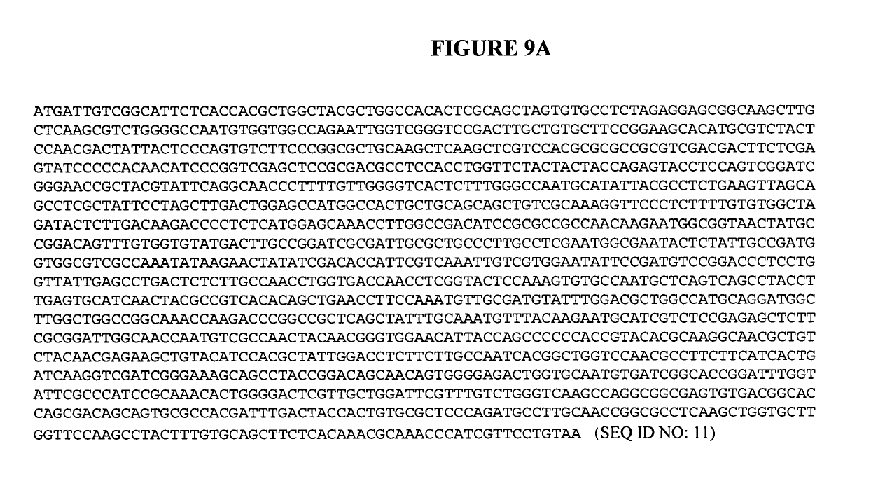

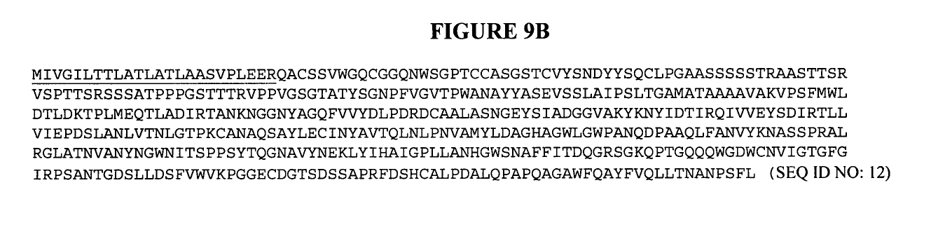

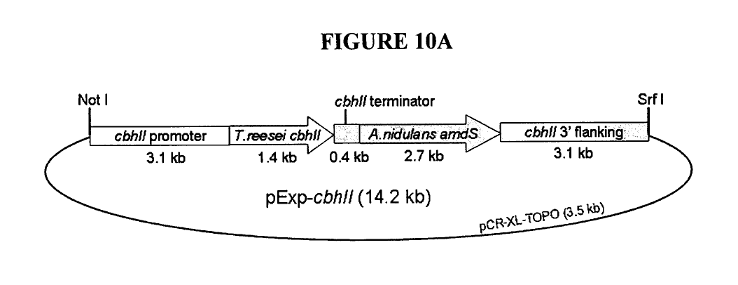

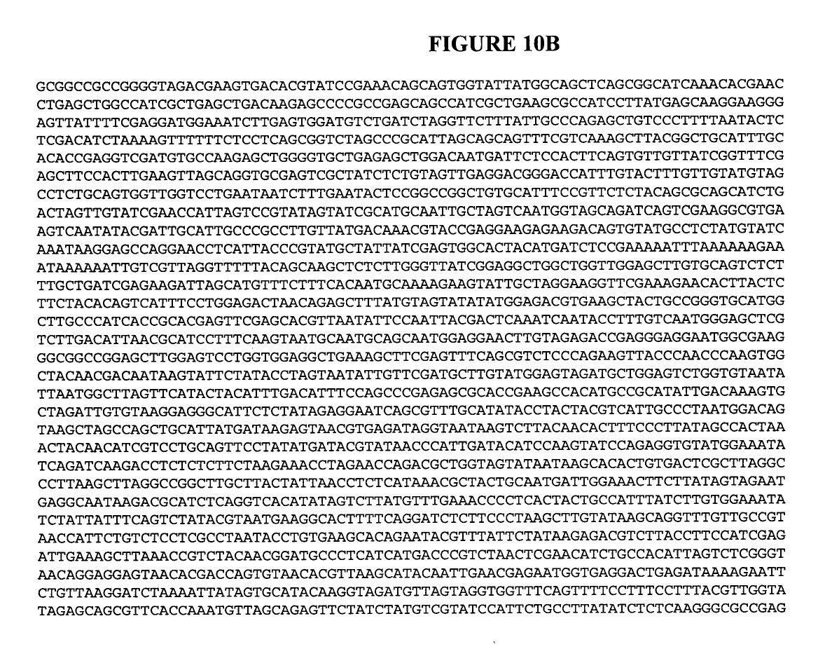

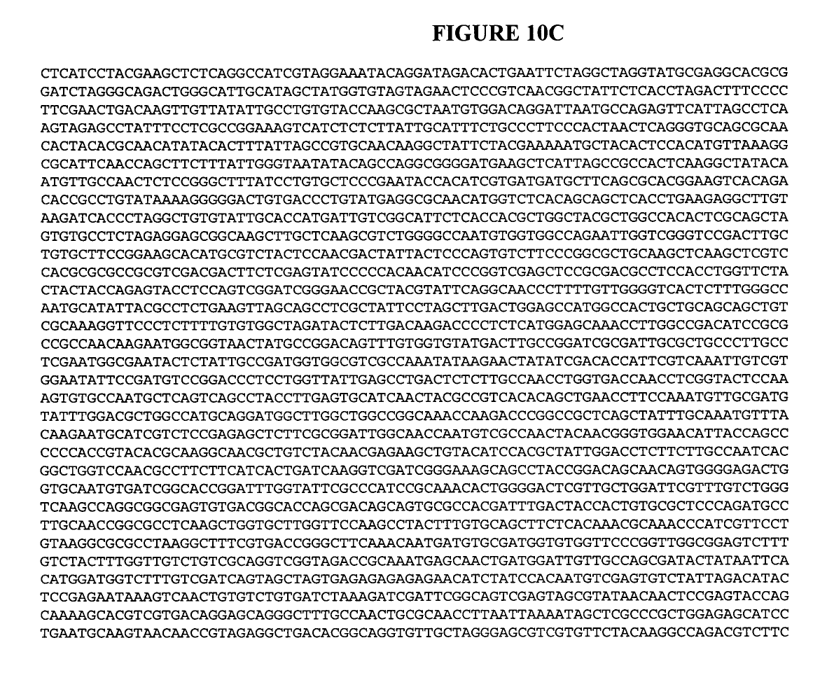

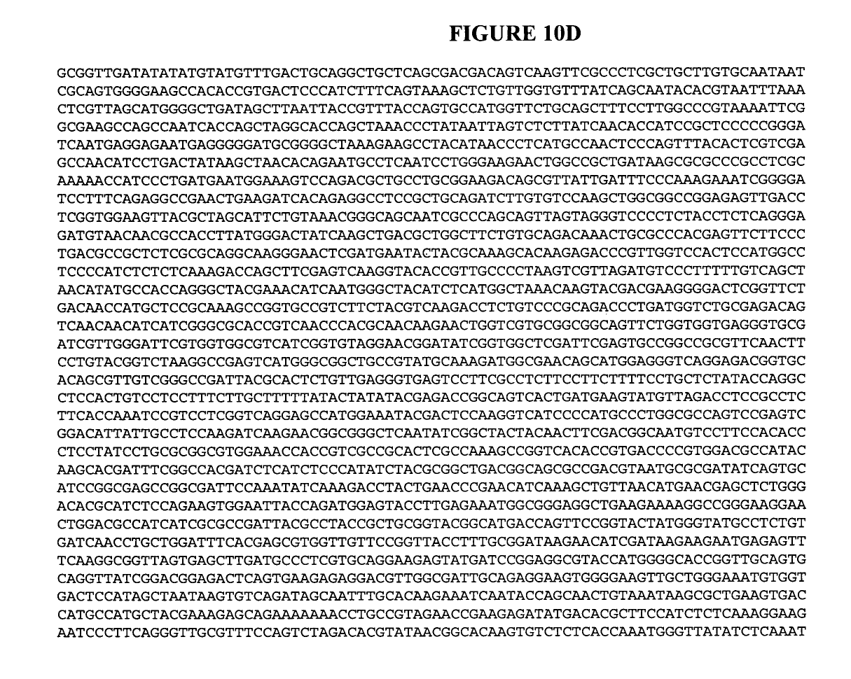

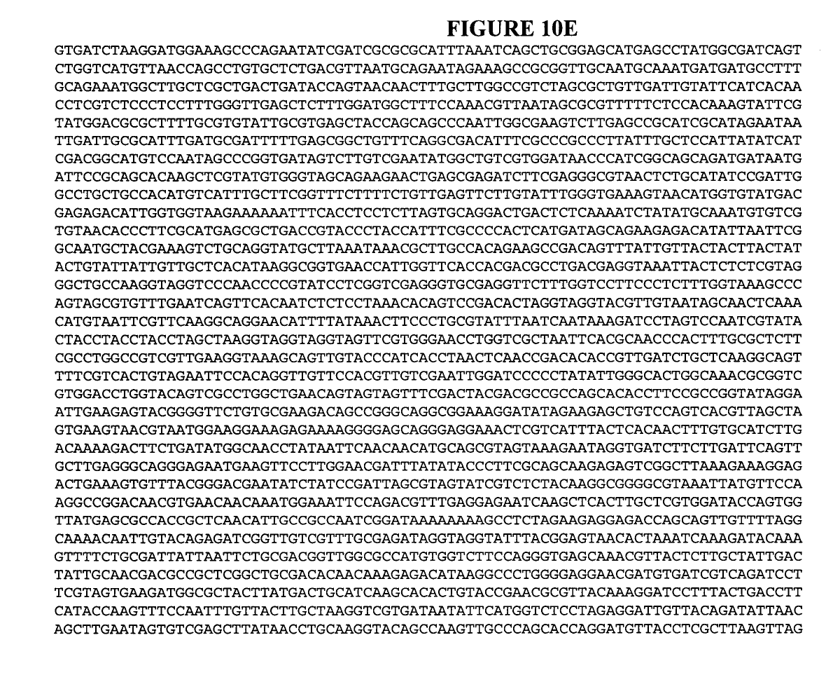

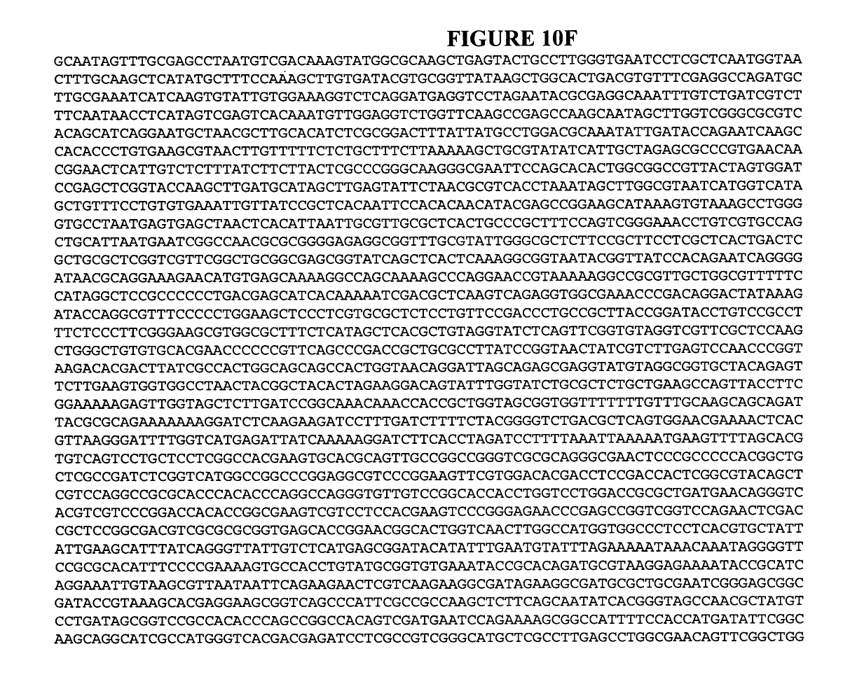

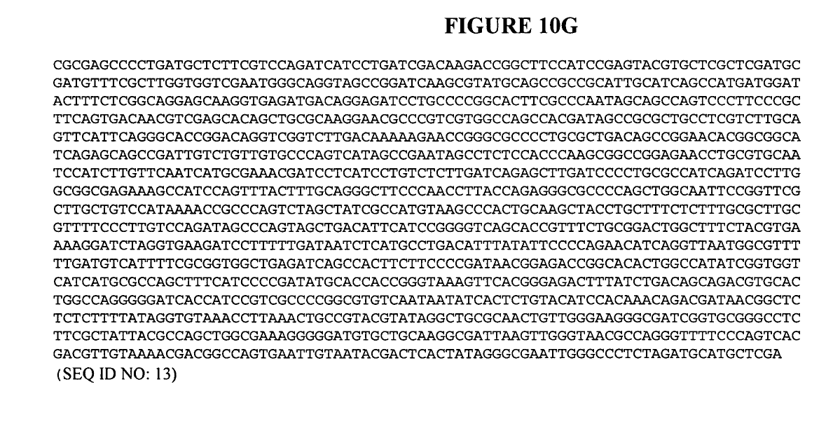

The vector expressed a T. reesei cbhII gene encoding an engineered protein with the following mutations in the amino acid sequence: P98L, M134V, T154A, I212V, S316P, and S413Y. The DNA sequence from start to stop codon was 1416 bases (SEQ ID NO: 11) as provided in FIG. 9A. The amino acid sequence (SEQ ID NO: 12) is provided in FIG. 9B (the signal sequence is underlined). A diagram of the cbhII expression vector is shown in FIG. 10A. The DNA sequence of the entire cbhII expression pExp-cbhII vector was 14158 bases (SEQ ID NO: 11) as provided in FIG. 10B.

Co-transformation was carried on a T. reesei strain deleted for cbhI, using three fragments of DNA:

The Engineered cbhII Expression Fragment that was Cut from the Plasmid pExp-cbhII using NotI and SrfI.

The engineered cbhI in the expression vector pTrex3g that was used as a PCR template to generate a linear fragment of only the cbhI promoter, engineered cbhI and cbhI terminator (without amdS marker). The cbhI-E1 fusion fragment described in the previous example that was used as a PCR template to generate a linear fragment consisting of the cbhI promoter, the cbhI-E1 fusion gene and cbhI terminator (without amdS marker). These three fragments were used to coat tungsten particles in biolistic cotransformation. The procedure was carried out as described in the previous example. In this cotransformation, each of the three fragments, 1, 2 and 3 were added to the tungsten particles at a volume of 1.5 μl of each fragment (100-300 ng/μl DNA concentration). Transformant selection was on MM acetamide media as described.

### 6.3 Example 3

### Assay of Cellulolytic Activity from Transformed Trichoderma reesei Clones

The following assays and substrates were used to determine the cellulolytic activity of the CBHI-E1 fusion protein. Trichoderma reesei strains Tr-A and Tr-D were derived from RL-P37 through mutagenesis.

Pretreated corn stover (PCS): Corn stover was pretreated with 2% w/w H2SO4 as described in Schell, D. et al., J. Appl. Biochem. Biotechnol. 105:69-86 (2003), and followed by multiple washes with deionized water to obtain a pH of 4.5. Sodium acetate was added to make a final concentration of 50 mM and this was titrated to pH 5.0.

Measurement of Total Protein: Protein concentrations were measured using the bicinchoninic acid method with bovine serum albumin as a standard (Smith, P. K., et al. (1985) Anal. Biochem. 150:76-85).

Cellulose conversion (Soluble sugar determinations) was evaluated by HPLC according to the methods described in Baker et al., Appl. Biochem. Biotechnol. 70-72:395-403 (1998).

A standard cellulosic conversion assay was used in the experiments. In this assay enzyme and buffered substrate were placed in containers and incubated at a temperature over time. The reaction was quenched with enough 100 mM Glycine, pH 11.0 to bring the pH of the reaction mixture to at least pH 10. Once the reaction was quenched, an aliquot of the reaction mixture was filtered through a 0.2 micron membrane to remove solids. The filtered solution was then assayed for soluble sugars by HPLC as described above. The cellulose concentration in the reaction mixture was approximately 7%. The enzyme or enzyme mixtures were dosed anywhere from 1 to 60 mg of total protein per gram of cellulose.

Table 1, below, summaries the data showing the increased specific performance of the 4-part strain over a modified Tr-D.

| mg/g | 4-part | Modified Tr-D |
|---|---|---|
| 10 | 9.5 | 5.1 |
| 20 | 14.2 | 8.1 |

PCS (13%) SSC, 20 hours, 65° C.

Table 2, below, summarizes the data showing the increased specific performance of the 3-part strain over Tr-A.

| mg/g | 3-part | Tr-A |
|---|---|---|
| 15 | 61 | 45 |
| 10 | 45 | 31 |

PCS (13%) SSC, 72 hours, 59° C.

All references and publications cited herein are incorporated by reference in their entirety. It should be noted that there are alternative ways of implementing the present invention. Accordingly, the present embodiments are to be considered as illustrative and not restrictive, and the invention is not to be limited to the details given herein, but may be modified within the scope and equivalents of the appended claims.

1. A filamentous fungus comprising
a first polynucleotide encoding a first heterologous polypeptide,
a second polynucleotide encoding a second heterologous polypeptide, and
a third polynucleotide encoding a homologous polypeptide
wherein the filamentous fungus is capable of expressing the first and second heterologous polypeptide and the homologous polypeptide and
wherein the first and second heterologous polypeptide and the homologous polypeptide form a functional mixture.

2. The filamentous fungus of claim 1, wherein the first polynucleotide is operably linked to a first promoter.

3. The filamentous fungus of claim 1, wherein the second polynucleotide is fused with the third polynucleotide and wherein the second and third polynucleotides are operably linked to a second promoter.

4. The filamentous fungus of claim 1, wherein the first polynucleotide is operably linked to a promoter native to the gene encoding the homologous polypeptide.

5. The filamentous fungus of claim 1, wherein the second polynucleotide is fused with the third polynucleotide and wherein the third polynucleotide is operably linked to a promoter of a gene encoding the homologous polypeptide.

6. The filamentous fungus of claim 1, wherein the second polynucleotide is fused with the third polynucleotide to form a polynucleotide encoding a fusion protein, wherein the fusion protein comprises the second heterologous polypeptide and the homologous polypeptide separated by a linker.

7. The filamentous fungus of claim 6, wherein the fusion protein further comprises a cleavage site.

8. The filamentous fungus of claim 1 further comprising a fourth polynucleotide encoding a selectable marker.

9. The filamentous fungus of claim 1 further comprising a fourth polynucleotide encoding a third heterologous polypeptide, wherein the filamentous fungus is capable of expressing the third heterologous polypeptide.

10. The filamentous fungus of claim 1, wherein the first heterologous polypeptide is a modified homologous polypeptide.

11. The filamentous fungus of claim 1 further comprising a fourth polynucleotide encoding a third heterologous polypeptide, wherein the first and second heterologous polypeptides are modified homologous polypeptides.

12. The filamentous fungus of claim 1, wherein the first heterologous polypeptide, the second heterologous polypeptide or the homologous polypeptide is an enzyme.

13. The filamentous fungus of claim 1, wherein the first heterologous polypeptide, the second heterologous polypeptide or the homologous polypeptide is a cellulase.

14. The filamentous fungus of claim 1, wherein the functional mixture is a mixture of cellulases.

15. The filamentous fungus of claim 1, wherein the first heterologous polypeptide, the second heterologous polypeptide or the homologous polypeptide is a cellulase selected from the group consisting of exo-cellobiohydrolases, endoglucanases, and beta-glucosidases.

16. The filamentous fungus of claim 1, wherein the first heterologous polypeptide is an exo-cellobiohydrolase and the second heterologous polypeptide is an endoglucanase.

17. The filamentous fungus of claim 1, wherein the first heterologous polypeptide is an exo-cellobiohydrolase selected from the group consisting of GH family 5, 6, 7, 9 and 48, and wherein the second heterologous polypeptide is an endoglucanase selected from the group consisting of GH family 5, 6, 7, 8, 9, 12, 17, 31, 44, 45, 48, 51, 61, 64, 74, and 81.

18. The filamentous fungus of claim 1, wherein the first heterologous polypeptide is an exo-cellobiohydrolase, the second heterologous polypeptide is an endoglucanase, and wherein the homologous polypeptide is an exo-cellobiohydrolase.

19. The filamentous fungus of claim 1, wherein the first heterologous polypeptide is a first exo-cellobiohydrolase, the second heterologous polypeptide is an endoglucanase, the homologous polypeptide is a second exo-cellobiohydrolase, and wherein the first exo-cellobiohydrolase and the second exo-cellobiohydrolase correspond to the same member of cellobiohydrolases.

20. The filamentous fungus of claim 1, wherein the filamentous fungus is selected from the group consisting of Aspergillus, Acremonium, Aureobasidium, Beauveria, Cephalosporium, Ceriporiopsis, Chaetomium, Paecilomyces, Chrysosporium, Claviceps, Cochiobolus, Cryptococcus, Cyathus, Endothia, Fusarium, Gilocladium, Humicola, Magnaporthe, Myceliophthora, Myrothecium, Mucor, Neurospora, Phanerochaete, Podospora, Paecilomyces, Penicillium, Pyricularia, Rhizomucor, Rhizopus, Schizophylum, Stagonospora, Talaromyces, Trichoderma, Thermomyces, Thermoascus, Thielavia, Tolypocladium, Trichophyton, Trametes, and Pleurotus.

21. The filamentous fungus of claim 1, wherein the filamentous fungus is T. reesei and wherein the first heterologous polypeptide is Humicola grisea CBHI, the second heterologous polypeptide is Acidothermus cellulolyticus endoglucanase 1, and wherein the homologous polypeptide is Trichoderma reesei CBHI.

22. The filamentous fungus of claim 1, wherein the filamentous fungus is T. reesei and wherein the first heterologous polypeptide or the second heterologous polypeptide is selected from the group consisting of Penicillium funiculosum cellobiohydrolase CBHI, Thermobifida endoglucanases E3, Thermobifida endoglucanases E5, Acidothermus cellulolyticus GH74-core and GH48.

23. The filamentous fungus of claim 1 further comprising a fourth polynucleotide encoding a third heterologous polypeptide, wherein the first polypeptide is a modified Trichoderma reesei CBHI, the second heterologous polypeptide is a modified Trichoderma reesei CBHII, the third heterologous polypeptide is Acidothermus cellulolyticus endoglucanase 1, and the homologous polypeptide is Trichoderma reesei CBHI.

24. The filamentous fungus of claim 1,
wherein the first heterologous polypeptide is an exo-cellobiohydrolase, the second heterologous polypeptide is an endoglucanase, and the homologous polypeptide is an exo-cellobiohydrolase, and
wherein expression of the first heterologous polypeptide, the second heterologous polypeptide and the homologous polypeptide forms a mixture of thermostable cellulases.

25. The filamentous fungus of claim 1, wherein the third polynucleotide is an extrachromosomal polynucleotide.

26. The filamentous fungus of claim 1, wherein the first, second, and third polynucleotide are extrachromosomal polynucleotides.

27. A culture medium comprising a population of the filamentous fungus of claim 1.

28. A polypeptide mixture comprising the first heterologous polypeptide, the second heterologous polypeptide, and the homologous polypeptide obtained from the filamentous fungus of claim 1.

29. The polypeptide mixture of claim 28, wherein the mixture is a mixture of cellulases.

30. A method of producing a mixture of cellulases comprising obtaining a polypeptide mixture from the filamentous fungus of claim 1, wherein the polypeptide mixture comprises the first heterologous polypeptide, the second heterologous polypeptide, and the homologous polypeptide.

31. A method of producing a mixture of cellulases comprising obtaining a polypeptide mixture from the filamentous fungus of claim 1,
wherein the polypeptide mixture comprises the first heterologous polypeptide, the second heterologous polypeptide, and the homologous polypeptide, and
wherein the first heterologous polypeptide is an exo-cellobiohydrolase, the second heterologous polypeptide is an endoglucanase, and the homologous polypeptide is an exo-cellobiohydrolase.

32. A method of producing a mixture of cellulases comprising obtaining a polypeptide mixture from the filamentous fungus of claim 1,
wherein the polypeptide mixture comprises the first heterologous polypeptide, the second heterologous polypeptide, and the homologous polypeptide,
wherein the first heterologous polypeptide is a first exo-cellobiohydrolase, the second heterologous polypeptide is an endoglucanase, the homologous polypeptide is a second exo-cellobiohydrolase, and
wherein the first exo-cellobiohydrolase and the second exo-cellobiohydrolase correspond to the same member of cellobiohydrolases.

33. A method of producing a mixture of cellulases comprising obtaining a polypeptide mixture from the filamentous fungus of claim 1,
wherein the polypeptide mixture comprises the first heterologous polypeptide, the second heterologous polypeptide, and the homologous polypeptide and wherein the filamentous fungus is T. reesei and the first heterologous polypeptide is Humicola grisea CBHI, the second heterologous polypeptide is Acidothermus cellulolyticus endoglucanase 1, and the homologous polypeptide is Trichoderma reesei CBHI.

34. A method of producing a mixture of cellulases comprising obtaining a polypeptide mixture from the filamentous fungus of claim 23, wherein the polypeptide mixture comprises the first heterologous polypeptide, the second heterologous polypeptide, the third heterologous polypeptide and the homologous polypeptide.

35. The filamentous fungus of claim 1, wherein the first heterologous polypeptide, the second heterologous polypeptide or the homologous polypeptide is a xylanase.

36. The filamentous fungus of claim 1, wherein the first heterologous polypeptide, the second heterologous polypeptide or the homologous polypeptide is an endoglucanase.

37. The filamentous fungus of claim 1, wherein the filamentous fungus expresses a GH 61 family member.

38. The filamentous fungus of claim 1, wherein the first heterologous polypeptide, the second heterologous polypeptide or the homologous polypeptide are each cellulases, and wherein each polypeptide is independently selected from the group consisting of exo-cellobiohydrolases, endoglucanases, and beta-glucosidases.

39. The filamentous fungus of claim 1, wherein the first heterologous polypeptide is a Trichoderma reesei CBHI, the second heterologous polypeptide is a Trichoderma reesei CBHII, and the homologous polypeptide is Trichoderma reesei CBHI.

40. The polypeptide mixture of claim 28, wherein the polypeptide mixture is a functional mixture.

41. The polypeptide mixture of claim 40 which does not include any bacterial enzyme in combination with its carrier filamentous protein and/or wherein the functional mixture does not form any antibody or functional antibody fragment.

42. The polypeptide mixture of claim 40 which displays an improved function of cellulase activity, saccharification activity, thermal stability, alter pH values, sustained activity for greater time periods at the same temperature.

43. The polypeptide mixture of claim 42, wherein the polypeptide mixture is a functional mixture that displays improved cellulase activity.

44. The polypeptide mixture of claim 42, wherein the polypeptide mixture is a functional mixture that displays improved saccharification activity.

45. A filamentous fungus comprising two or more heterologous polypeptides, and a homologous polypeptide, wherein the filmentous fungus is capable of expressing the heterologous polypeptides and the homologous polypeptides and wherein the heterologous polypeptides and the homologous polypeptide form a functional mixture.

46. The filamentous fungus of claim 45 which does not include any bacterial enzyme in combination with its carrier filamentous protein and/or wherein the functional mixture does not form any antibody or functional antibody fragment.

47. A recombinant filamentous fungus that is genetically modified to express a combination of heterologous and homologous polypeptides.

48. The recombinant filamentous fungus of claim 47 which produces a functional mixture.

49. The recombinant filamentous fungus of claim 47 that is genetically modified to express two or more heterologous polypeptides and a homologous polypeptide.

50. The recombinant filamentous fungus of claim 49 which produces a functional mixture.

51. The recombinant filamentous fungus of claim 50, wherein the functional mixture is a functional mixture of cellulases.

52. The recombinant filamentous fungus of claim 51, wherein the functional mixture has a function derived from two or three of the polypeptides from the mixture.

53. The recombinant filamentous fungus of claim 49 that is genetically modified to express three or more heterologous polypeptides and a homologous polypeptide.

54. The recombinant filamentous fungus of claim 53 which produces a functional mixture.

55. The recombinant filamentous fungus of claim 54, wherein the functional mixture is a functional mixture of cellulases.

56. The recombinant filamentous fungus of claim 55, wherein the functional mixture has a function derived from two or three of the polypeptides from the mixture.

57. The recombinant filamentous fungus of claim 49 that is genetically modified to express four or more heterologous polypeptides and a homologous polypeptide.

58. The recombinant filamentous fungus of claim 57 which produces a functional mixture.

59. The recombinant filamentous fungus of claim 58, wherein the functional mixture is a functional mixture of cellulases.

60. The recombinant filamentous fungus of claim 59, wherein the functional mixture has a function derived from two, three or four of the polypeptides from the mixture.

61. The recombinant filamentous fungus of claim 49, wherein the heterologous polypeptides and the homologous polypeptide are cellulases.

62. The recombinant filamentous fungus of claim 61, wherein each cellulase is independently selected from the group consisting of exo-cellobiohydrolases endoglucanases, and beta-glucosidases.

63. The recombinant filamentous fungus of claim 62 which is genetically modified to express an exo-cellobiohydrolase.

64. The recombinant filamentous fungus of claim 63 wherein the exo-cellobiohydrolase is a CBHI-type enzyme.

65. The recombinant filamentous fungus of claim 64, wherein the CBHI-type enzyme is a variant of H. jecorina CBHI.

66. The recombinant filamentous fungus of claim 63, wherein the exo-cellobiohydrolase is a CBHII-type enzyme.

67. The recombinant filamentous fungus of claim 66, wherein the CBHII-type enzyme is a variant of H. jecorina CBHII.

68. The recombinant filamentous fungus of claim 62 which is genetically modified to express an endoglucanase.

69. The recombinant filamentous fungus of claim 62 which is genetically modified to express a beta-glucosidase.

70. The recombinant filamentous fungus of claim 47 which is genetically modified to express a heterologous exo-cellobiohydrolase and a heterologous endoglucanase.

71. The recombinant filamentous fungus of claim 70, wherein the exo-cellobiohydrolase is a GH5, GH6, GH7, GH9 or GH48, and wherein the endoglucanase is a GH5, GH6, GH7, GH8, GH9, GH12, GH17, GH31, GH44, GH45, GH48, GH51, GH61, GH64, GH74 or GH81.

72. The recombinant filamentous fungus of claim 47, which is genetically modified to express a functional mixture of polypeptides selected from T. reesei EGI, T. reesei EGII, T. reesei EGIII, H. grisea EGIII, T. fusca E5, T. reesei E3, A. cellulolyticus E1 and T. reesei GH74.

73. The recombinant filamentous fungus of claim 49, wherein the heterologous polypeptides are an exo-cellobiohydrolase and an endoglucanase and wherein the homologous polypeptide is an exo-cellobiohydrolase.

74. The recombinant filamentous fungus of claim 49, wherein at least one heterologous polypeptide and at least one homologous polypeptide are expressed as a fusion polypeptide.

75. The recombinant filamentous fungus of claim 74, wherein said heterologous polypeptide and said homologous polypeptide are separated by a linker or a linker region, optionally wherein the linker is an Aspergillus glucoamylase linker or a Trichoderma CBHI linker.

76. The recombinant filamentous fungus of claim 74, wherein said heterologous polypeptide and said homologous polypeptide are separated by a linker or a linker region and a cleavage site, optionally wherein the cleavage site is a kexin cleavage site, a trypsin protease recognition site or an endoproteinase Lys-C recognition site.

77. The recombinant filamentous fungus of claim 45 which comprises a polynucleotide encoding a selectable marker, optionally wherein the selectable marker is an antimicrobial resistance marker, T. reesei pyr4, T. reesei acetolactate synthase, Streptomyces hyg, Aspergillus nidulans amdS or Aspergillus niger pyrG.

78. The recombinant filamentous fungus of claim 49, wherein at least one heterologous polypeptide and at least one homologous polypeptide are not expressed as a fusion polypeptide.

79. The recombinant filamentous fungus of claim 47, wherein the heterologous or homologous polypeptides are encoded by polynucleotides that are operably linked to one or more promoters.

80. The recombinant filamentous fungus of claim 79, wherein the polynucleotides are operably linked to one or more promoters native to the filamentous fungus.

81. The recombinant filamentous fungus of claim 79, wherein the polynucleotides are operably linked to one or more heterologous promoters.

82. The recombinant filamentous fungus of claim 79, wherein the polynucleotides are expressed under a constitutive promoter.

83. The recombinant filamentous fungus of claim 79, wherein the polynucleotides are expressed under an inducible promoter.

84. The recombinant filamentous fungus of claim 79, wherein the one or more promoters is selected from a cellulase promoter, a xylanase promoter, and the 1818 promoter.

85. The recombinant filamentous fungus of claim 79, wherein the one or more promoters is a cellulase promoter of the filamentous fungus.

86. The recombinant filamentous fungus of claim 85, wherein the cellulase promoter is an exo-cellobiohydrolase promoter, an endoglucanase promoter, or a beta-glucosidase promoter.

87. The recombinant filamentous fungus of claim 86, wherein the promoter is a cbh1 promoter.

88. The recombinant filamentous fungus of claim 79, wherein the one or more promoters is selected from a cbh1, cbh2, egl1, egl2, egl3, egl4, egl5, pkil, gpdl, xynl, or xyn2 promoter.

89. The recombinant filamentous fungus of claim 47 which is genetically modified to express a cellulase, a hemicellulase, a xylanase, or a mannanase.

90. The recombinant filamentous fungus of claim 47 which is genetically modified to express a GH5, GH6, GH7, GH9, or GH48 family member.

91. The recombinant filamentous fungus of claim 47 which is genetically modified to express a GH5, GH6, GH7, GH8, GH9, GH12, GH17, GH31, GH44, GH45, GH48, GH51, GH61, GH64, GH74 or GH81 family member.

92. The recombinant filamentous fungus of claim 91 which is genetically modified to express a GH61 family member.

93. The recombinant filamentous fungus of claim 47 which is genetically modified to express a GH1, GH3, GH9 or GH48 family member.

94. The recombinant filamentous fungus of any one of claims 47 to 93, which is selected from Aspergillus, Acremonium, Aureobasidium, Beauveria, Cephalosporium, Ceriporiopsis, Chaetomium, Paecilomyces, Chrysosporium, Claviceps, Cochiobolus, Cryptococcus, Cyathus, Endothia, Fusarium, Gilocladium, Humicola, Magnaporthe, Myceliophthora, Myrothecium, Mucor, Neurospora, Phanerochaete, Podospora, Paecilomyces, Penicillium, Pyricularia, Rhizomucor, Rhizopus, Schizophylum, Stagonospora, Talaromyces, Trichoderma, Thermomyces, Thermoascus, Thielavia, Tolypocladium, Trichophyton, Trametes, and Pleurotus.

95. A method of producing a combination of heterologous and homologous polypeptides, comprising culturing the recombinant filamentous fungus of claim 94.

96. A culture medium comprising a filamentous fungus according to 94.

97. A functional mixture comprising the heterologous and homologous polypeptides expressed by the recombinant filamentous fungus of any one of claims 48, 50, 54 and 58.

98. The functional mixture of claim 97 which displays an improved property and/or activity, or wherein the function of said functional mixture is an improved function with respect to an activity of, associated with, or provided by a filamentous fungus.

99. The functional mixture of claim 98 wherein the improved property, activity or function is improved cellulase activity, improved saccharification activity, improved thermal stability, an altered pH value, or a sustained activity for greater time periods at the same temperature.

100. The functional mixture of claim 99, wherein the improved property, activity or function is improved cellulase activity.

101. The functional mixture of claim 99, wherein the improved property, activity or function is improved saccharification activity.

102. The functional mixture of claim 99, which comprises cellulases, hemicellualses, xylanases, and mannanases.

103. The functional mixture of claim 99, which comprises a cellulase, hemicellualse, xylanase, or a mannanase.

104. The functional mixture of claim 99, which is a functional cellulase mixture.


SEQUENCE LISTING (14 sequences)

SEQ ID NO: 1 - DNA, 2656 bp, Artificial
  composite of Trichoderma reesei, Acidothermus cellulolyticus and synthetic sequences
atgtatcggaagttggccgtcatctcggccttcttggccacagctcgtgctcagtcggcctgcactctccaatcggagactcacccgcctctgacatggcagaaatgctcgtctggtggcacttgcactcaacagacaggctccgtggtcatcgacgccaactggcgctggactcacgctacgaacagcagcacgaactgctacgatggcaacacttggagctcgaccctatgtcctgacaacgagacctgcgcgaagaactgctgtctggacggtgccgcctacgcgtccacgtacggagttaccacgagcggtaacagcctctccattggctttgtcacccagtctgcgcagaagaacgttggcgctcgcctttaccttatggcgagcgacacgacctaccaggaattcaccctgcttggcaacgagttctctttcgatgttgatgtttcgcagctgccgtaagtgacttaccatgaacccctgacgtatcttcttgtgggctcccagctgactggccaatttaaggtgcggcttgaacggagctctctacttcgtgtccatggacgcggatggtggcgtgagcaagtatcccaccaacaccgctggcgccaagtacggcacggggtactgtgacagccagtgtccccgcgatctgaagttcatcaatggccaggccaacgttgagggctgggagccgtcatccaacaacgcaaacacgggcattggaggacacggaagctgctgctctgagatggatatctgggaggccaactccatctccgaggctcttaccccccacccttgcacgactgtcggccaggagatctgcgagggtgatgggtgcggcggaacttactccgataacagatatggcggcacttgcgatcccgatggctgcgactggaacccataccgcctgggcaacaccagcttctacggccctggctcaagctttaccctcgataccaccaagaaattgaccgttgtcacccagttcgagacgtcgggtgccatcaaccgatactatgtccagaatggcgtcactttccagcagcccaacgccgagcttggtagttactctggcaacgagctcaacgatgattactgcacagctgaggaggcagaattcggcggatcctctttctcagacaagggcggcctgactcagttcaagaaggctacctctggcggcatggttctggtcatgagtctgtgggatgatgtgagtttgatggacaaacatgcgcgttgacaaagagtcaagcagctgactgagatgttacagtactacgccaacatgctgtggctggactccacctacccgacaaacgagacctcctccacacccggtgccgtgcgcggaagctgctccaccagctccggtgtccctgctcaggtcgaatctcagtctcccaacgccaaggtcaccttctccaacatcaagttcggacccattggcagcaccggcaaccctagcggcggcaaccctcccggcggaaacccgcctggcaccaccaccacccgccgcccagccactaccactggaagctctcccggacctactagtaagcgggcgggcggcggctattggcacacgagcggccgggagatcctggacgcgaacaacgtgccggtacggatcgccggcatcaactggtttgggttcgaaacctgcaattacgtcgtgcacggtctctggtcacgcgactaccgcagcatgctcgaccagataaagtcgctcggctacaacacaatccggctgccgtactctgacgacattctcaagccgggcaccatgccgaacagcatcaatttttaccagatgaatcaggacctgcagggtctgacgtccttgcaggtcatggacaaaatcgtcgcgtacgccggtcagatcggcctgcgcatcattcttgaccgccaccgaccggattgcagcgggcagtcggcgctgtggtacacgagcagcgtctcggaggctacgtggatttccgacctgcaagcgctggcgcagcgctacaagggaaacccgacggtcgtcggctttgacttgcacaacgagccgcatgacccggcctgctggggctgcggcgatccgagcatcgactggcgattggccgccgagcgggccggaaacgccgtgctctcggtgaatccgaacctgctcattttcgtcgaaggtgtgcagagctacaacggagactcctactggtggggcggcaacctgcaaggagccggccagtacccggtcgtgctgaacgtgccgaaccgcctggtgtactcggcgcacgactacgcgacgagcgtctacccgcagacgtggttcagcgatccgaccttccccaacaacatgcccggcatctggaacaagaactggggatacctcttcaatcagaacattgcaccggtatggctgggcgaattcggtacgacactgcaatccacgaccgaccagacgtggctgaagacgctcgtccagtacctacggccgaccgcgcaatacggtgcggacagcttccagtggaccttctggtcctggaaccccgattccggcgacacaggaggaattctcaaggatgactggcagacggtcgacacagtaaaagacggctatctcgcgccgatcaagtcgtcgattttcgatcctgtcggctaa

SEQ ID NO: 2 - protein, 841 aa, Artificial
  composite of T. reesei, Aciothermus cellulyticus and synthetic sequences
MYRKLAVISAFLATARAQSACTLQSETHPPLTWQKCSSGGTCTQQTGSVVIDANWRWTHATNSSTNCYDGNTWSSTLCPDNETCAKNCCLDGAAYASTYGVTTSGNSLSIGFVTQSAQKNVGARLYLMASDTTYQEFTLLGNEFSFDVDVSQLPCGLNGALYFVSMDADGGVSKYPTNTAGAKYGTGYCDSQCPRDLKFINGQANVEGWEPSSNNANTGIGGHGSCCSEMDIWEANSISEALTPHPCTTVGQEICEGDGCGGTYSDNRYGGTCDPDGCDWNPYRLGNTSFYGPGSSFTLDTTKKLTVVTQFETSGAINRYYVQNGVTFQQPNAELGSYSGNELNDDYCTAEEAEFGGSSFSDKGGLTQFKKATSGGMVLVMSLWDDYYANMLWLDSTYPTNETSSTPGAVRGSCSTSSGVPAQVESQSPNAKVTFSNIKFGPIGSTGNPSGGNPPGGNPPGTTTTRRPATTTGSSPGPTSKRAGGGYWHTSGREILDANNVPVRIAGINWFGFETCNYVVHGLWSRDYRSMLDQIKSLGYNTIRLPYSDDILKPGTMPNSINFYQMNQDLQGLTSLQVMDKIVAYAGQIGLRIILDRHRPDCSGQSALWYTSSVSEATWISDLQALAQRYKGNPTVVGFDLHNEPHDPACWGCGDPSIDWRLAAERAGNAVLSVNPNLLIFVEGVQSYNGDSYWWGGNLQGAGQYPVVLNVPNRLVYSAHDYATSVYPQTWFSDPTFPNNMPGIWNKNWGYLFNQNIAPVWLGEFGTTLQSTTDQTWLKTLVQYLRPTAQYGADSFQWTFWSWNPDSGDTGGILKDDWQTVDTVKDGYLAPIKSSIFDPVG

SEQ ID NO: 3 - DNA, 41 bp, Artificial
  forward PCR primer
gcttatactagtaagcgcgcgggcggcggctattggcacac

SEQ ID NO: 4 - DNA, 39 bp, Artificial
  reverse PCR primer
gcttatggcgcgccttagacaggatcgaaaatcgacgac

SEQ ID NO: 5 - DNA, 24 bp, Artificial
  forward PCR primer
caccatgcgtaccgccaagttcgc

SEQ ID NO: 6 - DNA, 22 bp, Artificial
  reverse PCR primer
ttacaggcactgagagtaccag

SEQ ID NO: 7 - DNA, 10232 bp, Artificial
  pTrex3g-Hgrisea-cbh1 expression vector
aagcttactagtacttctcgagctctgtacatgtccggtcgcgacgtacgcgtatcgatggcgccagctgcaggcggccgcctgcagccacttgcagtcccgtggaattctcacggtgaatgtaggccttttgtagggtaggaattgtcactcaagcacccccaacctccattacgcctcccccatagagttcccaatcagtgagtcatggcactgttctcaaatagattggggagaagttgacttccgcccagagctgaaggtcgcacaaccgcatgatatagggtcggcaacggcaaaaaagcacgtggctcaccgaaaagcaagatgtttgcgatctaacatccaggaacctggatacatccatcatcacgcacgaccactttgatctgctggtaaactcgtattcgccctaaaccgaagtgcgtggtaaatctacacgtgggcccctttcggtatactgcgtgtgtcttctctaggtgccattcttttcccttcctctagtgttgaattgtttgtgttggagtccgagctgtaactacctctgaatctctggagaatggtggactaacgactaccgtgcacctgcatcatgtatataatagtgatcctgagaaggggggtttggagcaatgtgggactttgatggtcatcaaacaaagaacgaagacgcctcttttgcaaagttttgtttcggctacggtgaagaactggatacttgttgtgtcttctgtgtatttttgtggcaacaagaggccagagacaatctattcaaacaccaagcttgctcttttgagctacaagaacctgtggggtatatatctagagttgtgaagtcggtaatcccgctgtatagtaatacgagtcgcatctaaatactccgaagctgctgcgaacccggagaatcgagatgtgctggaaagcttctagcgagcggctaaattagcatgaaaggctatgagaaattctggagacggcttgttgaatcatggcgttccattcttcgacaagcaaagcgttccgtcgcagtagcaggcactcattcccgaaaaaactcggagattcctaagtagcgatggaaccggaataatataataggcaatacattgagttgcctcgacggttgcaatgcaggggtactgagcttggacataactgttccgtaccccacctcttctcaacctttggcgtttccctgattcagcgtacccgtacaagtcgtaatcactattaacccagactgaccggacgtgttttgcccttcatttggagaaataatgtcattgcgatgtgtaatttgcctgcttgaccgactggggctgttcgaagcccgaatgtaggattgttatccgaactctgctcgtagaggcatgttgtgaatctgtgtcgggcaggacacgcctcgaaggttcacggcaagggaaaccaccgatagcagtgtctagtagcaacctgtaaagccgcaatgcagcatcactggaaaatacaaaccaatggctaaaagtacataagttaatgcctaaagaagtcatataccagcggctaataattgtacaatcaagtggctaaacgtaccgtaatttgccaacggcttgtggggttgcagaagcaacggcaaagccccacttccccacgtttgtttcttcactcagtccaatctcagctggtgatcccccaattgggtcgcttgtttgttccggtgaagtgaaagaagacagaggtaagaatgtctgactcggagcgttttgcatacaaccaagggcagtgatggaagacagtgaaatgttgacattcaaggagtatttagccagggatgcttgagtgtatcgtgtaaggaggtttgtctgccgatacgacgaatactgtatagtcacttctgatgaagtggtccatattgaaatgtaaagtcggcactgaacaggcaaaagattgagttgaaactgcctaagatctcgggccctcgggccttcggcctttgggtgtacatgtttgtgctccgggcaaatgcaaagtgtggtaggatcgaacacactgctgcctttaccaagcagctgagggtatgtgataggcaaatgttcaggggccactgcatggtttcgaatagaaagagaagcttagccaagaacaatagccgataaagatagcctcattaaacggaatgagctagtaggcaaagtcagcgaatgtgtatatataaaggttcgaggtccgtgcctccctcatgctctccccatctactcatcaactcagatcctccaggagacttgtacaccatcttttgaggcacagaaacccaatagtcaaccatcacaagtttgtacaaaaaagcaggctatgcgtaccgccaagttcgccaccctcgccgcccttgtggcctcggccgccgcccagcaggcgtgcagtctcaccaccgagaggcacccttccctctcttggaagaagtgcaccgccggcggccagtgccagaccgtccaggcttccatcactctcgactccaactggcgctggactcaccaggtgtctggctccaccaactgctacacgggcaacaagtgggatactagcatctgcactgatgccaagtcgtgcgctcagaactgctgcgtcgatggtgccgactacaccagcacctatggcatcaccaccaacggtgattccctgagcctcaagttcgtcaccaagggccagcactcgaccaacgtcggctcgcgtacctacctgatggacggcgaggacaagtatcagagtacgttctatcttcagccttctcgcgccttgaatcctggctaacgtttacacttcacagccttcgagctcctcggcaacgagttcaccttcgatgtcgatgtctccaacatcggctgcggtctcaacggcgccctgtacttcgtctccatggacgccgatggtggtctcagccgctatcctggcaacaaggctggtgccaagtacggtaccggctactgcgatgctcagtgcccccgtgacatcaagttcatcaacggcgaggccaacattgagggctggaccggctccaccaacgaccccaacgccggcgcgggccgctatggtacctgctgctctgagatggatatctgggaagccaacaacatggctactgccttcactcctcacccttgcaccatcattggccagagccgctgcgagggcgactcgtgcggtggcacctacagcaacgagcgctacgccggcgtctgcgaccccgatggctgcgacttcaactcgtaccgccagggcaacaagaccttctacggcaagggcatgaccgtcgacaccaccaagaagatcactgtcgtcacccagttcctcaaggatgccaacggcgatctcggcgagatcaagcgcttctacgtccaggatggcaagatcatccccaactccgagtccaccatccccggcgtcgagggcaattccatcacccaggactggtgcgaccgccagaaggttgcctttggcgacattgacgacttcaaccgcaagggcggcatgaagcagatgggcaaggccctcgccggccccatggtcctggtcatgtccatctgggatgaccacgcctccaacatgctctggctcgactcgaccttccctgtcgatgccgctggcaagcccggcgccgagcgcggtgcctgcccgaccacctcgggtgtccctgctgaggttgaggccgaggcccccaacagcaacgtcgtcttctccaacatccgcttcggccccatcggctcgaccgttgctggtctccccggcgcgggcaacggcggcaacaacggcggcaaccccccgccccccaccaccaccacctcctcggctccggccaccaccaccaccgccagcgctggccccaaggctggccgctggcagcagtgcggcggcatcggcttcactggcccgacccagtgcgaggagccctacacttgcaccaagctcaacgactggtactctcagtgcctgtaaacccagctttcttgtacaaagtggtgatcgcgccagctccgtgcgaaagcctgacgcaccggtagattcttggtgagcccgtatcatgacggcggcgggagctacatggccccgggtgatttattttttttgtatctacttctgacccttttcaaatatacggtcaactcatctttcactggagatgcggcctgcttggtattgcgatgttgtcagcttggcaaattgtggctttcgaaaacacaaaacgattccttagtagccatgcattttaagataacggaatagaagaaagaggaaattaaaaaaaaaaaaaaaacaaacatcccgttcataacccgtagaatcgccgctcttcgtgtatcccagtaccagtttattttgaatagctcgcccgctggagagcatcctgaatgcaagtaacaaccgtagaggctgacacggcaggtgttgctagggagcgtcgtgttctacaaggccagacgtcttcgcggttgatatatatgtatgtttgactgcaggctgctcagcgacgacagtcaagttcgccctcgctgcttgtgcaataatcgcagtggggaagccacaccgtgactcccatctttcagtaaagctctgttggtgtttatcagcaatacacgtaatttaaactcgttagcatggggctgatagcttaattaccgtttaccagtgccatggttctgcagctttccttggcccgtaaaattcggcgaagccagccaatcaccagctaggcaccagctaaaccctataattagtctcttatcaacaccatccgctcccccgggatcaatgaggagaatgagggggatgcggggctaaagaagcctacataaccctcatgccaactcccagtttacactcgtcgagccaacatcctgactataagctaacacagaatgcctcaatcctgggaagaactggccgctgataagcgcgcccgcctcgcaaaaaccatccctgatgaatggaaagtccagacgctgcctgcggaagacagcgttattgatttcccaaagaaatcggggatcctttcagaggccgaactgaagatcacagaggcctccgctgcagatcttgtgtccaagctggcggccggagagttgacctcggtggaagttacgctagcattctgtaaacgggcagcaatcgcccagcagttagtagggtcccctctacctctcagggagatgtaacaacgccaccttatgggactatcaagctgacgctggcttctgtgcagacaaactgcgcccacgagttcttccctgacgccgctctcgcgcaggcaagggaactcgatgaatactacgcaaagcacaagagacccgttggtccactccatggcctccccatctctctcaaagaccagcttcgagtcaaggtacaccgttgcccctaagtcgttagatgtccctttttgtcagctaacatatgccaccagggctacgaaacatcaatgggctacatctcatggctaaacaagtacgacgaaggggactcggttctgacaaccatgctccgcaaagccggtgccgtcttctacgtcaagacctctgtcccgcagaccctgatggtctgcgagacagtcaacaacatcatcgggcgcaccgtcaacccacgcaacaagaactggtcgtgcggcggcagttctggtggtgagggtgcgatcgttgggattcgtggtggcgtcatcggtgtaggaacggatatcggtggctcgattcgagtgccggccgcgttcaacttcctgtacggtctaaggccgagtcatgggcggctgccgtatgcaaagatggcgaacagcatggagggtcaggagacggtgcacagcgttgtcgggccgattacgcactctgttgagggtgagtccttcgcctcttccttcttttcctgctctataccaggcctccactgtcctcctttcttgctttttatactatatacgagaccggcagtcactgatgaagtatgttagacctccgcctcttcaccaaatccgtcctcggtcaggagccatggaaatacgactccaaggtcatccccatgccctggcgccagtccgagtcggacattattgcctccaagatcaagaacggcgggctcaatatcggctactacaacttcgacggcaatgtccttccacaccctcctatcctgcgcggcgtggaaaccaccgtcgccgcactcgccaaagccggtcacaccgtgaccccgtggacgccatacaagcacgatttcggccacgatctcatctcccatatctacgcggctgacggcagcgccgacgtaatgcgcgatatcagtgcatccggcgagccggcgattccaaatatcaaagacctactgaacccgaacatcaaagctgttaacatgaacgagctctgggacacgcatctccagaagtggaattaccagatggagtaccttgagaaatggcgggaggctgaagaaaaggccgggaaggaactggacgccatcatcgcgccgattacgcctaccgctgcggtacggcatgaccagttccggtactatgggtatgcctctgtgatcaacctgctggatttcacgagcgtggttgttccggttacctttgcggataagaacatcgataagaagaatgagagtttcaaggcggttagtgagcttgatgccctcgtgcaggaagagtatgatccggaggcgtaccatggggcaccggttgcagtgcaggttatcggacggagactcagtgaagagaggacgttggcgattgcagaggaagtggggaagttgctgggaaatgtggtgactccatagctaataagtgtcagatagcaatttgcacaagaaatcaataccagcaactgtaaataagcgctgaagtgaccatgccatgctacgaaagagcagaaaaaaacctgccgtagaaccgaagagatatgacacgcttccatctctcaaaggaagaatcccttcagggttgcgtttccagtctagacacgtataacggcacaagtgtctctcaccaaatgggttatatctcaaatgtgatctaaggatggaaagcccagaatatcgatcgcgcgcagatccatatatagggcccgggttataattacctcaggtcgacgtcccatggccattcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccc

SEQ ID NO: 8 - DNA, 1545 bp, Artificial
  engineered sequence based on T. reesei
atgtatcggaagttggccgtcatctcggccttcttggccacagctcgtgctcagtcggcctgcactcttcaaccggagactcacccgcctctgacatggcagaaatgctcgtctggtggcacgtgcactcaacagacaggctccgtggtcatcgacgccaactggcgctggattcacgctacgaacagcagcacgagctgctacgatggcaacacttggagctcgaccctatgtcctgacaacgagacctgcacgaagaactgctgtctggacggtgccgcctacgcgtccacgtacggagttaccacgagcggtgacagcctcaccattggctttgtcacccagtctgcgcagaagaacgttggcgctcgcctttaccttatggcgaacgacacgacctaccaggaattcaccctgcttggcaacgagttctctttcgatgttgatgtttcgcagctgccgtgcggcttgaacggagctctctacttcgtgtccatggacgcggatggtggcgtgagcaagtatcccaccaacaccgctggcgccaagtacggcacggggtactgtgacagccagtgtccccgcgatctgaagttcatcaatggccaggccaacgttgagggctgggagccgtcaaccaacaacgcgaacacgggcattggaggacacggaagctgctgctctgagatggatatctgggaggccaactctatctccgaggctcttaccctccacccttgcacgactgtcggccaggagatctgcgagggtgatgggtgcggcggaacttactccaagaacagatatggcggcccttgcgatcccgatggctgcgactggaacccataccgcctgggcaacaccagcttctacggccctggcccaagctttaccctcgataccaccaagaaattgaccgttgtcacccagttcaagccgtcgggtgccatcaaccgatactatgtccagaatggcgtcactttccagcagcccaacgccgagcttggtagttactctggcaacgagctcaacgatgattactgctacgctgaggaggcagaattcggcggatcctctttctcagacaagggcggcctgactcagttcaagaaggctacctctggcggcatggttctggtcatgagtctgtgggatgattactacgccaacatgctgtggctggactccacctacccgacaaacgagacctcctccacacccggtgccgtgcgcggaagctgctccaccagctccggtgaccctgctcaggtcgaatctcagtttcccaacgccaaggtcaccttctccaacatcaagttcggacccattggcagcaccggcaaccctagcggcggcaaccctcccggcggaaacccgcctggcaccaccaccacccgccgcccagccactaccactggaagctctcccggacctacccagtctcactacggccagtgcggcggtattggctacagcggccccacggtctgcgccagcggcacaacttgccaggtcctgaacccttactactctcagtgcctgtaa

SEQ ID NO: 9 - protein, 514 aa, Artificial
  engineered sequence based on T. reesei
MYRKLAVISAFLATARAQSACTLQPETHPPLTWQKCSSGGTCTQQTGSVVIDANWRWIHATNSSTSCYDGNTWSSTLCPDNETCTKNCCLDGAAYASTYGVTTSGDSLTIGFVTQSAQKNVGARLYLMANDTTYQEFTLLGNEFSFDVDVSQLPCGLNGALYFVSMDADGGVSKYPTNTAGAKYGTGYCDSQCPRDLKFINGQANVEGWEPSTNNANTGIGGHGSCCSEMDIWEANSISEALTLHPCTTVGQEICEGDGCGGTYSKNRYGGPCDPDGCDWNPYRLGNTSFYGPGPSFTLDTTKKLTVVTQFKPSGAINRYYVQNGVTFQQPNAELGSYSGNELNDDYCYAEEAEFGGSSFSDKGGLTQFKKATSGGMVLVMSLWDDYYANMLWLDSTYPTNETSSTPGAVRGSCSTSSGDPAQVESQFPNAKVTFSNIKFGPIGSTGNPSGGNPPGGNPPGTTTTRRPATTTGSSPGPTQSHYGQCGGIGYSGPTVCASGTTCQVLNPYYSQCL

SEQ ID NO: 10 - DNA, 10145 bp, Artificial
  pTrex3g-cbh1 expression vector
aagcttactagtacttctcgagctctgtacatgtccggtcgcgacgtacgcgtatcgatggcgccagctgcaggcggccgcctgcagccacttgcagtcccgtggaattctcacggtgaatgtaggccttttgtagggtaggaattgtcactcaagcacccccaacctccattacgcctcccccatagagttcccaatcagtgagtcatggcactgttctcaaatagattggggagaagttgacttccgcccagagctgaaggtcgcacaaccgcatgatatagggtcggcaacggcaaaaaagcacgtggctcaccgaaaagcaagatgtttgcgatctaacatccaggaacctggatacatccatcatcacgcacgaccactttgatctgctggtaaactcgtattcgccctaaaccgaagtgcgtggtaaatctacacgtgggcccctttcggtatactgcgtgtgtcttctctaggtgccattcttttcccttcctctagtgttgaattgtttgtgttggagtccgagctgtaactacctctgaatctctggagaatggtggactaacgactaccgtgcacctgcatcatgtatataatagtgatcctgagaaggggggtttggagcaatgtgggactttgatggtcatcaaacaaagaacgaagacgcctcttttgcaaagttttgtttcggctacggtgaagaactggatacttgttgtgtcttctgtgtatttttgtggcaacaagaggccagagacaatctattcaaacaccaagcttgctcttttgagctacaagaacctgtggggtatatatctagagttgtgaagtcggtaatcccgctgtatagtaatacgagtcgcatctaaatactccgaagctgctgcgaacccggagaatcgagatgtgctggaaagcttctagcgagcggctaaattagcatgaaaggctatgagaaattctggagacggcttgttgaatcatggcgttccattcttcgacaagcaaagcgttccgtcgcagtagcaggcactcattcccgaaaaaactcggagattcctaagtagcgatggaaccggaataatataataggcaatacattgagttgcctcgacggttgcaatgcaggggtactgagcttggacataactgttccgtaccccacctcttctcaacctttggcgtttccctgattcagcgtacccgtacaagtcgtaatcactattaacccagactgaccggacgtgttttgcccttcatttggagaaataatgtcattgcgatgtgtaatttgcctgcttgaccgactggggctgttcgaagcccgaatgtaggattgttatccgaactctgctcgtagaggcatgttgtgaatctgtgtcgggcaggacacgcctcgaaggttcacggcaagggaaaccaccgatagcagtgtctagtagcaacctgtaaagccgcaatgcagcatcactggaaaatacaaaccaatggctaaaagtacataagttaatgcctaaagaagtcatataccagcggctaataattgtacaatcaagtggctaaacgtaccgtaatttgccaacggcttgtggggttgcagaagcaacggcaaagccccacttccccacgtttgtttcttcactcagtccaatctcagctggtgatcccccaattgggtcgcttgtttgttccggtgaagtgaaagaagacagaggtaagaatgtctgactcggagcgttttgcatacaaccaagggcagtgatggaagacagtgaaatgttgacattcaaggagtatttagccagggatgcttgagtgtatcgtgtaaggaggtttgtctgccgatacgacgaatactgtatagtcacttctgatgaagtggtccatattgaaatgtaaagtcggcactgaacaggcaaaagattgagttgaaactgcctaagatctcgggccctcgggccttcggcctttgggtgtacatgtttgtgctccgggcaaatgcaaagtgtggtaggatcgaacacactgctgcctttaccaagcagctgagggtatgtgataggcaaatgttcaggggccactgcatggtttcgaatagaaagagaagcttagccaagaacaatagccgataaagatagcctcattaaacggaatgagctagtaggcaaagtcagcgaatgtgtatatataaaggttcgaggtccgtgcctccctcatgctctccccatctactcatcaactcagatcctccaggagacttgtacaccatcttttgaggcacagaaacccaatagtcaaccatcacaagtttgtacaaaaaacaggctatgtatcggaagttggccgtcatctcggccttcttggccacagctcgtgctcagtcggcctgcactcttcaaccggagactcacccgcctctgacatggcagaaatgctcgtctggtggcacgtgcactcaacagacaggctccgtggtcatcgacgccaactggcgctggattcacgctacgaacagcagcacgagctgctacgatggcaacacttggagctcgaccctatgtcctgacaacgagacctgcacgaagaactgctgtctggacggtgccgcctacgcgtccacgtacggagttaccacgagcggtgacagcctcaccattggctttgtcacccagtctgcgcagaagaacgttggcgctcgcctttaccttatggcgaacgacacgacctaccaggaattcaccctgcttggcaacgagttctctttcgatgttgatgtttcgcagctgccgtgcggcttgaacggagctctctacttcgtgtccatggacgcggatggtggcgtgagcaagtatcccaccaacaccgctggcgccaagtacggcacggggtactgtgacagccagtgtccccgcgatctgaagttcatcaatggccaggccaacgttgagggctgggagccgtcaaccaacaacgcgaacacgggcattggaggacacggaagctgctgctctgagatggatatctgggaggccaactctatctccgaggctcttaccctccacccttgcacgactgtcggccaggagatctgcgagggtgatgggtgcggcggaacttactccaagaacagatatggcggcccttgcgatcccgatggctgcgactggaacccataccgcctgggcaacaccagcttctacggccctggcccaagctttaccctcgataccaccaagaaattgaccgttgtcacccagttcaagccgtcgggtgccatcaaccgatactatgtccagaatggcgtcactttccagcagcccaacgccgagcttggtagttactctggcaacgagctcaacgatgattactgctacgctgaggaggcagaattcggcggatcctctttctcagacaagggcggcctgactcagttcaagaaggctacctctggcggcatggttctggtcatgagtctgtgggatgattactacgccaacatgctgtggctggactccacctacccgacaaacgagacctcctccacacccggtgccgtgcgcggaagctgctccaccagctccggtgaccctgctcaggtcgaatctcagtttcccaacgccaaggtcaccttctccaacatcaagttcggacccattggcagcaccggcaaccctagcggcggcaaccctcccggcggaaacccgcctggcaccaccaccacccgccgcccagccactaccactggaagctctcccggacctacccagtctcactacggccagtgcggcggtattggctacagcggccccacggtctgcgccagcggcacaacttgccaggtcctgaacccttactactctcagtgcctgtaaacccagctttcttgtacaaagtggtgatcgcgccgcgcgccagctccgtgcgaaagcctgacgcaccggtagattcttggtgagcccgtatcatgacggcggcgggagctacatggccccgggtgatttattttttttgtatctacttctgacccttttcaaatatacggtcaactcatctttcactggagatgcggcctgcttggtattgcgatgttgtcagcttggcaaattgtggctttcgaaaacacaaaacgattccttagtagccatgcattttaagataacggaatagaagaaagaggaaattaaaaaaaaaaaaaaaacaaacatcccgttcataacccgtagaatcgccgctcttcgtgtatcccagtaccagtttattttgaatagctcgcccgctggagagcatcctgaatgcaagtaacaaccgtagaggctgacacggcaggtgttgctagggagcgtcgtgttctacaaggccagacgtcttcgcggttgatatatatgtatgtttgactgcaggctgctcagcgacgacagtcaagttcgccctcgctgcttgtgcaataatcgcagtggggaagccacaccgtgactcccatctttcagtaaagctctgttggtgtttatcagcaatacacgtaatttaaactcgttagcatggggctgatagcttaattaccgtttaccagtgccatggttctgcagctttccttggcccgtaaaattcggcgaagccagccaatcaccagctaggcaccagctaaaccctataattagtctcttatcaacaccatccgctcccccgggatcaatgaggagaatgagggggatgcggggctaaagaagcctacataaccctcatgccaactcccagtttacactcgtcgagccaacatcctgactataagctaacacagaatgcctcaatcctgggaagaactggccgctgataagcgcgcccgcctcgcaaaaaccatccctgatgaatggaaagtccagacgctgcctgcggaagacagcgttattgatttcccaaagaaatcggggatcctttcagaggccgaactgaagatcacagaggcctccgctgcagatcttgtgtccaagctggcggccggagagttgacctcggtggaagttacgctagcattctgtaaacgggcagcaatcgcccagcagttagtagggtcccctctacctctcagggagatgtaacaacgccaccttatgggactatcaagctgacgctggcttctgtgcagacaaactgcgcccacgagttcttccctgacgccgctctcgcgcaggcaagggaactcgatgaatactacgcaaagcacaagagacccgttggtccactccatggcctccccatctctctcaaagaccagcttcgagtcaaggtacaccgttgcccctaagtcgttagatgtccctttttgtcagctaacatatgccaccagggctacgaaacatcaatgggctacatctcatggctaaacaagtacgacgaaggggactcggttctgacaaccatgctccgcaaagccggtgccgtcttctacgtcaagacctctgtcccgcagaccctgatggtctgcgagacagtcaacaacatcatcgggcgcaccgtcaacccacgcaacaagaactggtcgtgcggcggcagttctggtggtgagggtgcgatcgttgggattcgtggtggcgtcatcggtgtaggaacggatatcggtggctcgattcgagtgccggccgcgttcaacttcctgtacggtctaaggccgagtcatgggcggctgccgtatgcaaagatggcgaacagcatggagggtcaggagacggtgcacagcgttgtcgggccgattacgcactctgttgagggtgagtccttcgcctcttccttcttttcctgctctataccaggcctccactgtcctcctttcttgctttttatactatatacgagaccggcagtcactgatgaagtatgttagacctccgcctcttcaccaaatccgtcctcggtcaggagccatggaaatacgactccaaggtcatccccatgccctggcgccagtccgagtcggacattattgcctccaagatcaagaacggcgggctcaatatcggctactacaacttcgacggcaatgtccttccacaccctcctatcctgcgcggcgtggaaaccaccgtcgccgcactcgccaaagccggtcacaccgtgaccccgtggacgccatacaagcacgatttcggccacgatctcatctcccatatctacgcggctgacggcagcgccgacgtaatgcgcgatatcagtgcatccggcgagccggcgattccaaatatcaaagacctactgaacccgaacatcaaagctgttaacatgaacgagctctgggacacgcatctccagaagtggaattaccagatggagtaccttgagaaatggcgggaggctgaagaaaaggccgggaaggaactggacgccatcatcgcgccgattacgcctaccgctgcggtacggcatgaccagttccggtactatgggtatgcctctgtgatcaacctgctggatttcacgagcgtggttgttccggttacctttgcggataagaacatcgataagaagaatgagagtttcaaggcggttagtgagcttgatgccctcgtgcaggaagagtatgatccggaggcgtaccatggggcaccggttgcagtgcaggttatcggacggagactcagtgaagagaggacgttggcgattgcagaggaagtggggaagttgctgggaaatgtggtgactccatagctaataagtgtcagatagcaatttgcacaagaaatcaataccagcaactgtaaataagcgctgaagtgaccatgccatgctacgaaagagcagaaaaaaacctgccgtagaaccgaagagatatgacacgcttccatctctcaaaggaagaatcccttcagggttgcgtttccagtctagacacgtataacggcacaagtgtctctcaccaaatgggttatatctcaaatgtgatctaaggatggaaagcccagaatatcgatcgcgcgcagatccatatatagggcccgggttataattacctcaggtcgacgtcccatggccattcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccc

SEQ ID NO: 11 - DNA, 1416 bp, Artificial
  engineered sequence based on T. reesei
atgattgtcggcattctcaccacgctggctacgctggccacactcgcagctagtgtgcctctagaggagcggcaagcttgctcaagcgtctggggccaatgtggtggccagaattggtcgggtccgacttgctgtgcttccggaagcacatgcgtctactccaacgactattactcccagtgtcttcccggcgctgcaagctcaagctcgtccacgcgcgccgcgtcgacgacttctcgagtatcccccacaacatcccggtcgagctccgcgacgcctccacctggttctactactaccagagtacctccagtcggatcgggaaccgctacgtattcaggcaacccttttgttggggtcactctttgggccaatgcatattacgcctctgaagttagcagcctcgctattcctagcttgactggagccatggccactgctgcagcagctgtcgcaaaggttccctcttttgtgtggctagatactcttgacaagacccctctcatggagcaaaccttggccgacatccgcgccgccaacaagaatggcggtaactatgccggacagtttgtggtgtatgacttgccggatcgcgattgcgctgcccttgcctcgaatggcgaatactctattgccgatggtggcgtcgccaaatataagaactatatcgacaccattcgtcaaattgtcgtggaatattccgatgtccggaccctcctggttattgagcctgactctcttgccaacctggtgaccaacctcggtactccaaagtgtgccaatgctcagtcagcctaccttgagtgcatcaactacgccgtcacacagctgaaccttccaaatgttgcgatgtatttggacgctggccatgcaggatggcttggctggccggcaaaccaagacccggccgctcagctatttgcaaatgtttacaagaatgcatcgtctccgagagctcttcgcggattggcaaccaatgtcgccaactacaacgggtggaacattaccagccccccaccgtacacgcaaggcaacgctgtctacaacgagaagctgtacatccacgctattggacctcttcttgccaatcacggctggtccaacgccttcttcatcactgatcaaggtcgatcgggaaagcagcctaccggacagcaacagtggggagactggtgcaatgtgatcggcaccggatttggtattcgcccatccgcaaacactggggactcgttgctggattcgtttgtctgggtcaagccaggcggcgagtgtgacggcaccagcgacagcagtgcgccacgatttgactaccactgtgcgctcccagatgccttgcaaccggcgcctcaagctggtgcttggttccaagcctactttgtgcagcttctcacaaacgcaaacccatcgttcctgtaa

SEQ ID NO: 12 - protein, 471 aa, Artificial
  engineered sequence based on T. reesei
MIVGILTTLATLATLAASVPLEERQACSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAYYASEVSSLAIPSLTGAMATAAAAVAKVPSFMWLDTLDKTPLMEQTLADIRTANKNGGNYAGQFVVYDLPDRDCAALASNGEYSIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGPLLANHGWSNAFFITDQGRSGKQPTGQQQWGDWCNVIGTGFGIRPSANTGDSLLDSFVWVKPGGECDGTSDSSAPRFDSHCALPDALQPAPQAGAWFQAYFVQLLTNANPSFL

SEQ ID NO: 13 - DNA, 14158 bp, Artificial
  expression vector pExp-cbhII
gcggccgccggggtagacgaagtgacacgtatccgaaacagcagtggtattatggcagctcagcggcatcaaacacgaacctgagctggccatcgctgagctgacaagagccccgccgagcagccatcgctgaagcgccatccttatgagcaaggaagggagttattttcgaggatggaaatcttgagtggatgtctgatctaggttctttattgcccagagctgtcccttttaatactctcgacatctaaaagttttttctcctcagcggtctagcccgcattagcagcagtttcgtcaaagcttacggctgcatttgcacaccgaggtcgatgtgccaagagctggggtgctgagagctggacaatgattctccacttcagtgttgttatcggtttcgagcttccacttgaagttagcaggtgcgagtcgctatctctgtagttgaggacgggaccatttgtactttgttgtatgtagcctctgcagtggttggtcctgaataatctttgaatactccggccggctgtgcatttccgttctctacagcgcagcatctgactagttgtatcgaaccattagtccgtatagtatcgcatgcaattgctagtcaatggtagcagatcagtcgaaggcgtgaagtcaatatacgattgcattgcccgccttgttatgacaaacgtaccgaggaagagaagacagtgtatgcctctatgtatcaaataaggagccaggaacctcattacccgtatgctattatcgagtggcactacatgatctccgaaaaatttaaaaaagaaataaaaaattgtcgttaggtttttacagcaagctctcttgggttatcggaggctggctggttggagcttgtgcagtctctttgctgatcgagaagattagcatgtttctttcacaatgcaaaagaagtattgctaggaaggttcgaaagaacacttactcttctacacagtcatttcctggagactaacagagctttatgtagtatatatggagacgtgaagctactgccgggtgcatggcttgcccatcaccgcacgagttcgagcacgttaatattccaattacgactcaaatcaatacctttgtcaatgggagctcgtcttgacattaacgcatcctttcaagtaatgcaatgcagcaatggaggaacttgtagagaccgagggaggaatggcgaagggcggccggagcttggagtcctggtggaggctgaaagcttcgagtttcagcgtctcccagaagttacccaacccaagtggctacaacgacaataagtattctatacctagtaatattgttcgatgcttgtatggagtagatgctggagtctggtgtaatattaatggcttagttcatactacatttgacatttccagcccgagagcgcaccgaagccacatgccgcatattgacaaagtgctagattgtgtaaggagggcattctctatagaggaatcagcgtttgcatatacctactacgtcattgccctaatggacagtaagctagccagctgcattatgataagagtaacgtgagataggtaataagtcttacaacactttcccttatagccactaaactacaacatcgtcctgcagttcctatatgatacgtataacccattgatacatccaagtatccagaggtgtatggaaatatcagatcaagacctctctcttctaagaaacctagaaccagacgctggtagtataataagcacactgtgactcgcttaggcccttaagcttaggccggcttgcttactattaacctctcataaacgctactgcaatgattggaaacttcttatagtagaatgaggcaataagacgcatctcaggtcacatatagtcttatgtttgaaacccctcactactgccatttatcttgtggaaatatctattatttcagtctatacgtaatgaaggcacttttcaggatctcttccctaagcttgtataagcaggtttgttgccgtaaccattctgtctcctcgcctaatacctgtgaagcacagaatacgtttattctataagagacgtcttaccttccatcgagattgaaagcttaaaccgtctacaacggatgccctcatcatgacccgtctaactcgaacatctgccacattagtctcgggtaacaggaggagtaacacgaccagtgtaacacgttaagcatacaattgaacgagaatggtgaggactgagataaaagaattctgttaaggatctaaaattatagtgcatacaaggtagatgttagtaggtggtttcagttttcctttcctttacgttggtatagagcagcgttcaccaaatgttagcagagttctatctatgtcgtatccattctgccttatatctctcaagggcgccgagctcatcctacgaagctctcaggccatcgtaggaaatacaggatagacactgaattctaggctaggtatgcgaggcacgcggatctagggcagactgggcattgcatagctatggtgtagtagaactcccgtcaacggctattctcacctagactttccccttcgaactgacaagttgttatattgcctgtgtaccaagcgctaatgtggacaggattaatgccagagttcattagcctcaagtagagcctatttcctcgccggaaagtcatctctcttattgcatttctgcccttcccactaactcagggtgcagcgcaacactacacgcaacatatacactttattagccgtgcaacaaggctattctacgaaaaatgctacactccacatgttaaaggcgcattcaaccagcttctttattgggtaatatacagccaggcggggatgaagctcattagccgccactcaaggctatacaatgttgccaactctccgggctttatcctgtgctcccgaataccacatcgtgatgatgcttcagcgcacggaagtcacagacaccgcctgtataaaagggggactgtgaccctgtatgaggcgcaacatggtctcacagcagctcacctgaagaggcttgtaagatcaccctaggctgtgtattgcaccatgattgtcggcattctcaccacgctggctacgctggccacactcgcagctagtgtgcctctagaggagcggcaagcttgctcaagcgtctggggccaatgtggtggccagaattggtcgggtccgacttgctgtgcttccggaagcacatgcgtctactccaacgactattactcccagtgtcttcccggcgctgcaagctcaagctcgtccacgcgcgccgcgtcgacgacttctcgagtatcccccacaacatcccggtcgagctccgcgacgcctccacctggttctactactaccagagtacctccagtcggatcgggaaccgctacgtattcaggcaacccttttgttggggtcactctttgggccaatgcatattacgcctctgaagttagcagcctcgctattcctagcttgactggagccatggccactgctgcagcagctgtcgcaaaggttccctcttttgtgtggctagatactcttgacaagacccctctcatggagcaaaccttggccgacatccgcgccgccaacaagaatggcggtaactatgccggacagtttgtggtgtatgacttgccggatcgcgattgcgctgcccttgcctcgaatggcgaatactctattgccgatggtggcgtcgccaaatataagaactatatcgacaccattcgtcaaattgtcgtggaatattccgatgtccggaccctcctggttattgagcctgactctcttgccaacctggtgaccaacctcggtactccaaagtgtgccaatgctcagtcagcctaccttgagtgcatcaactacgccgtcacacagctgaaccttccaaatgttgcgatgtatttggacgctggccatgcaggatggcttggctggccggcaaaccaagacccggccgctcagctatttgcaaatgtttacaagaatgcatcgtctccgagagctcttcgcggattggcaaccaatgtcgccaactacaacgggtggaacattaccagccccccaccgtacacgcaaggcaacgctgtctacaacgagaagctgtacatccacgctattggacctcttcttgccaatcacggctggtccaacgccttcttcatcactgatcaaggtcgatcgggaaagcagcctaccggacagcaacagtggggagactggtgcaatgtgatcggcaccggatttggtattcgcccatccgcaaacactggggactcgttgctggattcgtttgtctgggtcaagccaggcggcgagtgtgacggcaccagcgacagcagtgcgccacgatttgactaccactgtgcgctcccagatgccttgcaaccggcgcctcaagctggtgcttggttccaagcctactttgtgcagcttctcacaaacgcaaacccatcgttcctgtaaggcgcgcctaaggctttcgtgaccgggcttcaaacaatgatgtgcgatggtgtggttcccggttggcggagtctttgtctactttggttgtctgtcgcaggtcggtagaccgcaaatgagcaactgatggattgttgccagcgatactataattcacatggatggtctttgtcgatcagtagctagtgagagagagagaacatctatccacaatgtcgagtgtctattagacatactccgagaataaagtcaactgtgtctgtgatctaaagatcgattcggcagtcgagtagcgtataacaactccgagtaccagcaaaagcacgtcgtgacaggagcagggctttgccaactgcgcaaccttaattaaaatagctcgcccgctggagagcatcctgaatgcaagtaacaaccgtagaggctgacacggcaggtgttgctagggagcgtcgtgttctacaaggccagacgtcttcgcggttgatatatatgtatgtttgactgcaggctgctcagcgacgacagtcaagttcgccctcgctgcttgtgcaataatcgcagtggggaagccacaccgtgactcccatctttcagtaaagctctgttggtgtttatcagcaatacacgtaatttaaactcgttagcatggggctgatagcttaattaccgtttaccagtgccatggttctgcagctttccttggcccgtaaaattcggcgaagccagccaatcaccagctaggcaccagctaaaccctataattagtctcttatcaacaccatccgctcccccgggatcaatgaggagaatgagggggatgcggggctaaagaagcctacataaccctcatgccaactcccagtttacactcgtcgagccaacatcctgactataagctaacacagaatgcctcaatcctgggaagaactggccgctgataagcgcgcccgcctcgcaaaaaccatccctgatgaatggaaagtccagacgctgcctgcggaagacagcgttattgatttcccaaagaaatcggggatcctttcagaggccgaactgaagatcacagaggcctccgctgcagatcttgtgtccaagctggcggccggagagttgacctcggtggaagttacgctagcattctgtaaacgggcagcaatcgcccagcagttagtagggtcccctctacctctcagggagatgtaacaacgccaccttatgggactatcaagctgacgctggcttctgtgcagacaaactgcgcccacgagttcttccctgacgccgctctcgcgcaggcaagggaactcgatgaatactacgcaaagcacaagagacccgttggtccactccatggcctccccatctctctcaaagaccagcttcgagtcaaggtacaccgttgcccctaagtcgttagatgtccctttttgtcagctaacatatgccaccagggctacgaaacatcaatgggctacatctcatggctaaacaagtacgacgaaggggactcggttctgacaaccatgctccgcaaagccggtgccgtcttctacgtcaagacctctgtcccgcagaccctgatggtctgcgagacagtcaacaacatcatcgggcgcaccgtcaacccacgcaacaagaactggtcgtgcggcggcagttctggtggtgagggtgcgatcgttgggattcgtggtggcgtcatcggtgtaggaacggatatcggtggctcgattcgagtgccggccgcgttcaacttcctgtacggtctaaggccgagtcatgggcggctgccgtatgcaaagatggcgaacagcatggagggtcaggagacggtgcacagcgttgtcgggccgattacgcactctgttgagggtgagtccttcgcctcttccttcttttcctgctctataccaggcctccactgtcctcctttcttgctttttatactatatacgagaccggcagtcactgatgaagtatgttagacctccgcctcttcaccaaatccgtcctcggtcaggagccatggaaatacgactccaaggtcatccccatgccctggcgccagtccgagtcggacattattgcctccaagatcaagaacggcgggctcaatatcggctactacaacttcgacggcaatgtccttccacaccctcctatcctgcgcggcgtggaaaccaccgtcgccgcactcgccaaagccggtcacaccgtgaccccgtggacgccatacaagcacgatttcggccacgatctcatctcccatatctacgcggctgacggcagcgccgacgtaatgcgcgatatcagtgcatccggcgagccggcgattccaaatatcaaagacctactgaacccgaacatcaaagctgttaacatgaacgagctctgggacacgcatctccagaagtggaattaccagatggagtaccttgagaaatggcgggaggctgaagaaaaggccgggaaggaactggacgccatcatcgcgccgattacgcctaccgctgcggtacggcatgaccagttccggtactatgggtatgcctctgtgatcaacctgctggatttcacgagcgtggttgttccggttacctttgcggataagaacatcgataagaagaatgagagtttcaaggcggttagtgagcttgatgccctcgtgcaggaagagtatgatccggaggcgtaccatggggcaccggttgcagtgcaggttatcggacggagactcagtgaagagaggacgttggcgattgcagaggaagtggggaagttgctgggaaatgtggtgactccatagctaataagtgtcagatagcaatttgcacaagaaatcaataccagcaactgtaaataagcgctgaagtgaccatgccatgctacgaaagagcagaaaaaaacctgccgtagaaccgaagagatatgacacgcttccatctctcaaaggaagaatcccttcagggttgcgtttccagtctagacacgtataacggcacaagtgtctctcaccaaatgggttatatctcaaatgtgatctaaggatggaaagcccagaatatcgatcgcgcgcatttaaatcagctgcggagcatgagcctatggcgatcagtctggtcatgttaaccagcctgtgctctgacgttaatgcagaatagaaagccgcggttgcaatgcaaatgatgatgcctttgcagaaatggcttgctcgctgactgataccagtaacaactttgcttggccgtctagcgctgttgattgtattcatcacaacctcgtctccctcctttgggttgagctctttggatggctttccaaacgttaatagcgcgtttttctccacaaagtattcgtatggacgcgcttttgcgtgtattgcgtgagctaccagcagcccaattggcgaagtcttgagccgcatcgcatagaataattgattgcgcatttgatgcgatttttgagcggctgtttcaggcgacatttcgcccgcccttatttgctccattatatcatcgacggcatgtccaatagcccggtgatagtcttgtcgaatatggctgtcgtggataacccatcggcagcagatgataatgattccgcagcacaagctcgtatgtgggtagcagaagaactgagcgagatcttcgagggcgtaactctgcatatccgattggcctgctgccacatgtcatttgcttcggtttcttttctgttgagttcttgtatttgggtgaaagtaacatggtgtatgacgagagacattggtggtaagaaaaaatttcacctcctcttagtgcaggactgactctcaaaatctatatgcaaatgtgtcgtgtaacacccttcgcatgagcgctgaccgtaccctaccatttcgccccactcatgatagcagaagagacatattaattcggcaatgctacgaaagtctgcaggtatgcttaaataaacgcttgccacagaagccgacagtttattgttactacttactatactgtattattgttgctcacataaggcggtgaaccattggttcaccacgacgcctgacgaggtaaattactctctcgtagggctgccaaggtaggtcccaaccccgtatcctcggtcgagggtgcgaggttctttggtccttccctctttggtaaagcccagtagcgtgtttgaatcagttcacaatctctcctaaacacagtccgacactaggtaggtacgttgtaatagcaactcaaacatgtaattcgttcaaggcaggaacattttataaacttccctgcgtatttaatcaataaagatcctagtccaatcgtatactacctacctacctagctaaggtaggtaggtagttcgtgggaacctggtcgctaattcacgcaacccactttgcgctcttcgcctggccgtcgttgaaggtaaagcagttgtacccatcacctaactcaaccgacacaccgttgatctgctcaaggcagttttcgtcactgtagaattccacaggttgttccacgttgtcgaattggatccccctatattgggcactggcaaacgcggtcgtggacctggtacagtcgcctggctgaacagtagtagtttcgactacgacgccgccagcacaccttccgccggtataggaattgaagagtacggggttctgtgcgaagacagccgggcaggcggaaaggatatagaagagctgtccagtcacgttagctagtgaagtaacgtaatggaaggaaagagaaaaggggagcagggaggaaactcgtcatttactcacaactttgtgcatcttgacaaaagacttctgatatggcaacctataattcaacaacatgcagcgtagtaaagaataggtgatcttcttgattcagttgcttgagggcagggagaatgaagttccttggaacgatttatatacccttcgcagcaagagagtcggcttaaagaaaggagactgaaagtgtttacgggacgaatatctatccgattagcgtagtatcgtctctacaaggcggggcgtaaattatgttccaaggccggacaacgtgaacaacaaatggaaattccagacgtttgaggagaatcaagctcacttgctcgtggataccagtggttatgagcgccaccgctcaacattgccgccaatcggataaaaaaaagcctctagaagaggagaccagcagttgttttaggcaaaacaattgtacagagatcggttgtcgtttgcgagataggtaggtatttacggagtaacactaaatcaaagatacaaagttttctgcgattattaattctgcgacggttggcgccatgtggtcttccagggtgagcaaacgttactcttgctattgactattgcaacgacgccgctcggctgcgacacaacaaagagacataaggccctggggaggaacgatgtgatcgtcagatccttcgtagtgaagatggcgctacttatgactgcatcaagcacactgtaccgaacgcgttacaaaggatcctttactgaccttcataccaagtttccaatttgttacttgctaaggtcgtgataatattcatggtctcctagaggattgttacagatattaacagcttgaatagtgtcgagcttataacctgcaaggtacagccaagttgcccagcaccaggatgttacctcgcttaagttaggcaatagtttgcgagcctaatgtcgacaaagtatggcgcaagctgagtactgccttgggtgaatcctcgctcaatggtaactttgcaagctcatatgctttccaaagcttgtgatacgtgcggttataagctggcactgacgtgtttcgaggccagatgcttgcgaaatcatcaagtgtattgtggaaaggtctcaggatgaggtcctagaatacgcgaggcaaatttgtctgatcgtctttcaataacctcatagtcgagtcacaaatgttggaggtctggttcaagccgagccaagcaatagcttggtcgggcgcgtcacagcatcaggaatgctaacgcttgcacatctcgcggactttattatgcctggacgcaaatattgataccagaatcaagccacaccctgtgaagcgtaacttgtttttctctgctttcttaaaaagctgcgtatatcattgctagagcgcccgtgaacaacggaactcattgtctctttatcttcttactcgcccgggcaagggcgaattccagcacactggcggccgttactagtggatccgagctcggtaccaagcttgatgcatagcttgagtattctaacgcgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaagcccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttagcacgtgtcagtcctgctcctcggccacgaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatggtggccctcctcacgtgctattattgaagcatttatcagggttattgtctcatgagcggatacatatttgaatgtatttagaaaaataaacaaataggggttccgcgcacatttccccgaaaagtgccacctgtatgcggtgtgaaataccgcacagatgcgtaaggagaaaataccgcatcaggaaattgtaagcgttaataattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcagatccttggcggcgagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgaaaaggatctaggtgaagatcctttttgataatctcatgcctgacatttatattccccagaacatcaggttaatggcgtttttgatgtcattttcgcggtggctgagatcagccacttcttccccgataacggagaccggcacactggccatatcggtggtcatcatgcgccagctttcatccccgatatgcaccaccgggtaaagttcacgggagactttatctgacagcagacgtgcactggccagggggatcaccatccgtcgccccggcgtgtcaataatatcactctgtacatccacaaacagacgataacggctctctcttttataggtgtaaaccttaaactgccgtacgtataggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggccctctagatgcatgctcga

SEQ ID NO: 14 - DNA, 11312 bp, Artificial
  pTrex4CBH1-E1 expression vector
aagcttaactagtacttctcgagctctgtacatgtccggtcgcgacgtacgcgtatcgatggcgccagctgcaggcggccgcctgcagccacttgcagtcccgtggaattctcacggtgaatgtaggccttttgtagggtaggaattgtcactcaagcacccccaacctccattacgcctcccccatagagttcccaatcagtgagtcatggcactgttctcaaatagattggggagaagttgacttccgcccagagctgaaggtcgcacaaccgcatgatatagggtcggcaacggcaaaaaagcacgtggctcaccgaaaagcaagatgtttgcgatctaacatccaggaacctggatacatccatcatcacgcacgaccactttgatctgctggtaaactcgtattcgccctaaaccgaagtgacgtggtaaatctacacgtgggcccctttcggtatactgcgtgtgtcttctctaggtgccattcttttcccttcctctagtgttgaattgtttgtgttggagtccgagctgtaactacctctgaatctctggagaatggtggactaacgactaccgtgcacctgcatcatgtatataatagtgatcctgagaaggggggtttggagcaatgtgggactttgatggtcatcaaacaaagaacgaagacgcctcttttgcaaagttttgtttcggctacggtgaagaactggatacttgttgtgtcttctgtgtatttttgtggcaacaagaggccagagacaatctattcaaacaccaagcttgctcttttgagctacaagaacctgtggggtatatatctagagttgtgaagtcggtaatcccgctgtatagtaatacgagtcgcatctaaatactccgaagctgctgcgaacccggagaatcgagatgtgctggaaagcttctagcgagcggctaaattagcatgaaaggctatgagaaattctggagacggcttgttgaatcatggcgttccattcttcgacaagcaaagcgttccgtcgcagtagcaggcactcattcccgaaaaaactcggagattcctaagtagcgatggaaccggaataatataataggcaatacattgagttgcctcgacggttgcaatgcaggggtactgagcttggacataactgttccgtaccccacctcttctcaacctttggcgtttccctgattcagcgtacccgtacaagtcgtaatcactattaacccagactgaccggacgtgttttgcccttcatttggagaaataatgtcattgcgatgtgtaatttgcctgcttgaccgactggggctgttcgaagcccgaatgtaggattgttatccgaactctgctcgtagaggcatgttgtgaatctgtgtcgggcaggacacgcctcgaaggttcacggcaagggaaaccaccgatagcagtgtctagtagcaacctgtaaagccgcaatgcagcatcactggaaaatacaaaccaatggctaaaagtacataagttaatgcctaaagaagtcatataccagcggctaataattgtacaatcaagtggctaaacgtaccgtaatttgccaacggcttgtggggttgcagaagcaacggcaaagccccacttccccacgtttgtttcttcactcagtccaatctcagctggtgatcccccaattgggtcgcttgtttgttccggtgaagtgaaagaagacagaggtaagaatgtctgactcggagcgttttgcatacaaccaagggcagtgatggaagacagtgaaatgttgacattcaaggagtatttagccagggatgcttgagtgtatcgtgtaaggaggtttgtctgccgatacgacgaatactgtatagtcacttctgatgaagtggtccatattgaaatgtaagtcggcactgaacaggcaaaagattgagttgaaactgcctaagatctcgggccctcgggccttcggcctttgggtgtacatgtttgtgctccgggcaaatgcaaagtgtggtaggatcgaacacactgctgcctttaccaagcagctgagggtatgtgataggcaaatgttcaggggccactgcatggtttcgaatagaaagagaagcttagccaagaacaatagccgataaagatagcctcattaaacggaatgagctagtaggcaaagtcagcgaatgtgtatatataaaggttcgaggtccgtgcctccctcatgctctccccatctactcatcaactcagatcctccaggagacttgtacaccatcttttgaggcacagaaacccaatagtcaaccgcggactgcgcatcatgtatcggaagttggccgtcatctcggccttcttggccacagctcgtgctcagtcggcctgcactctccaatcggagactcacccgcctctgacatggcagaaatgctcgtctggtggcacttgcactcaacagacaggctccgtggtcatcgacgccaactggcgctggactcacgctacgaacagcagcacgaactgctacgatggcaacacttggagctcgaccctatgtcctgacaacgagacctgcgcgaagaactgctgtctggacggtgccgcctacgcgtccacgtacggagttaccacgagcggtaacagcctctccattggctttgtcacccagtctgcgcagaagaacgttggcgctcgcctttaccttatggcgagcgacacgacctaccaggaattcaccctgcttggcaacgagttctctttcgatgttgatgtttcgcagctgccgtaagtgacttaccatgaacccctgacgtatcttcttgtgggctcccagctgactggccaatttaaggtgcggcttgaacggagctctctacttcgtgtccatggacgcggatggtggcgtgagcaagtatcccaccaacaccgctggcgccaagtacggcacggggtactgtgacagccagtgtccccgcgatctgaagttcatcaatggccaggccaacgttgagggctgggagccgtcatccaacaacgcaaacacgggcattggaggacacggaagctgctgctctgagatggatatctgggaggccaactccatctccgaggctcttaccccccacccttgcacgactgtcggccaggagatctgcgagggtgatgggtgcggcggaacttactccgataacagatatggcggcacttgcgatcccgatggctgcgactggaacccataccgcctgggcaacaccagcttctacggccctggctcaagctttaccctcgataccaccaagaaattgaccgttgtcacccagttcgagacgtcgggtgccatcaaccgatactatgtccagaatggcgtcactttccagcagcccaacgccgagcttggtagttactctggcaacgagctcaacgatgattactgcacagctgaggaggcagaattcggcggatcctctttctcagacaagggcggcctgactcagttcaagaaggctacctctggcggcatggttctggtcatgagtctgtgggatgatgtgagtttgatggacaaacatgcgcgttgacaaagagtcaagcagctgactgagatgttacagtactacgccaacatgctgtggctggactccacctacccgacaaacgagacctcctccacacccggtgccgtgcgcggaagctgctccaccagctccggtgtccctgctcaggtcgaatctcagtctcccaacgccaaggtcaccttctccaacatcaagttcggacccattggcagcaccggcaaccctagcggcggcaaccctcccggcggaaacccgcctggcaccaccaccacccgccgcccagccactaccactggaagctctcccggacctactagtaagcgggcgggcggcggctattggcacacgagcggccgggagatcctggacgcgaacaacgtgccggtacggatcgccggcatcaactggtttgggttcgaaacctgcaattacgtcgtgcacggtctctggtcacgcgactaccgcagcatgctcgaccagataaagtcgctcggctacaacacaatccggctgccgtactctgacgacattctcaagccgggcaccatgccgaacagcatcaatttttaccagatgaatcaggacctgcagggtctgacgtccttgcaggtcatggacaaaatcgtcgcgtacgccggtcagatcggcctgcgcatcattcttgaccgccaccgaccggattgcagcgggcagtcggcgctgtggtacacgagcagcgtctcggaggctacgtggatttccgacctgcaagcgctggcgcagcgctacaagggaaacccgacggtcgtcggctttgacttgcacaacgagccgcatgacccggcctgctggggctgcggcgatccgagcatcgactggcgattggccgccgagcgggccggaaacgccgtgctctcggtgaatccgaacctgctcattttcgtcgaaggtgtgcagagctacaacggagactcctactggtggggcggcaacctgcaaggagccggccagtacccggtcgtgctgaacgtgccgaaccgcctggtgtactcggcgcacgactacgcgacgagcgtctacccgcagacgtggttcagcgatccgaccttccccaacaacatgcccggcatctggaacaagaactggggatacctcttcaatcagaacattgcaccggtatggctgggcgaattcggtacgacactgcaatccacgaccgaccagacgtggctgaagacgctcgtccagtacctacggccgaccgcgcaatacggtgcggacagcttccagtggaccttctggtcctggaaccccgattccggcgacacaggaggaattctcaaggatgactggcagacggtcgacacagtaaaagacggctatctcgcgccgatcaagtcgtcgattttcgatcctgtctaaggcgcgccgcgcgccagctccgtgcgaaagcctgacgcaccggtagattcttggtgagcccgtatcatgacggcggcgggagctacatggccccgggtgatttattttttttgtatctacttctgacccttttcaaatatacggtcaactcatctttcactggagatgcggcctgcttggtattgcgatgttgtcagcttggcaaattgtggctttcgaaaacacaaaacgattccttagtagccatgcattttaagataacggaatagaagaaagaggaaattaaaaaaaaaaaaaaaacaaacatcccgttcataacccgtagaatcgccgctcttcgtgtatcccagtaccagtttattttgaatagctcgcccgctggagagcatcctgaatgcaagtaacaaccgtagaggctgacacggcaggtgttgctagggagcgtcgtgttctacaaggccagacgtcttcgcggttgatatatatgtatgtttgactgcaggctgctcagcgacgacagtcaagttcgccctcgctgcttgtgcaataatcgcagtggggaagccacaccgtgactcccatctttcagtaaagctctgttggtgtttatcagcaatacacgtaatttaaactcgttagcatggggctgatagcttaattaccgtttaccagtgccgcggttctgcagctttccttggcccgtaaaattcggcgaagccagccaatcaccagctaggcaccagctaaaccctataattagtctcttatcaacaccatccgctcccccgggatcaatgaggagaatgagggggatgcggggctaaagaagcctacataaccctcatgccaactcccagtttacactcgtcgagccaacatcctgactataagctaacacagaatgcctcaatcctgggaagaactggccgctgataagcgcgcccgcctcgcaaaaaccatccctgatgaatggaaagtccagacgctgcctgcggaagacagcgttattgatttcccaaagaaatcggggatcctttcagaggccgaactgaagatcacagaggcctccgctgcagatcttgtgtccaagctggcggccggagagttgacctcggtggaagttacgctagcattctgtaaacgggcagcaatcgcccagcagttagtagggtcccctctacctctcagggagatgtaacaacgccaccttatgggactatcaagctgacgctggcttctgtgcagacaaactgcgcccacgagttcttccctgacgccgctctcgcgcaggcaagggaactcgatgaatactacgcaaagcacaagagacccgttggtccactccatggcctccccatctctctcaaagaccagcttcgagtcaaggtacaccgttgcccctaagtcgttagatgtccctttttgtcagctaacatatgccaccagggctacgaaacatcaatgggctacatctcatggctaaacaagtacgacgaaggggactcggttctgacaaccatgctccgcaaagccggtgccgtcttctacgtcaagacctctgtcccgcagaccctgatggtctgcgagacagtcaacaacatcatcgggcgcaccgtcaacccacgcaacaagaactggtcgtgcggcggcagttctggtggtgagggtgcgatcgttgggattcgtggtggcgtcatcggtgtaggaacggatatcggtggctcgattcgagtgccggccgcgttcaacttcctgtacggtctaaggccgagtcatgggcggctgccgtatgcaaagatggcgaacagcatggagggtcaggagacggtgcacagcgttgtcgggccgattacgcactctgttgagggtgagtccttcgcctcttccttcttttcctgctctataccaggcctccactgtcctcctttcttgctttttatactatatacgagaccggcagtcactgatgaagtatgttagacctccgcctcttcaccaaatccgtcctcggtcaggagccatggaaatacgactccaaggtcatccccatgccctggcgccagtccgagtcggacattattgcctccaagatcaagaacggcgggctcaatatcggctactacaacttcgacggcaatgtccttccacaccctcctatcctgcgcggcgtggaaaccaccgtcgccgcactcgccaaagccggtcacaccgtgaccccgtggacgccatacaagcacgatttcggccacgatctcatctcccatatctacgcggctgacggcagcgccgacgtaatgcgcgatatcagtgcatccggcgagccggcgattccaaatatcaaagacctactgaacccgaacatcaaagctgttaacatgaacgagctctgggacacgcatctccagaagtggaattaccagatggagtaccttgagaaatggcgggaggctgaagaaaaggccgggaaggaactggacgccatcatcgcgccgattacgcctaccgctgcggtacggcatgaccagttccggtactatgggtatgcctctgtgatcaacctgctggatttcacgagcgtggttgttccggttacctttgcggataagaacatcgataagaagaatgagagtttcaaggcggttagtgagcttgatgccctcgtgcaggaagagtatgatccggaggcgtaccatggggcaccggttgcagtgcaggttatcggacggagactcagtgaagagaggacgttggcgattgcagaggaagtggggaagttgctgggaaatgtggtgactccatagctaataagtgtcagatagcaatttgcacaagaaatcaataccagcaactgtaaataagcgctgaagtgaccatgccatgctacgaaagagcagaaaaaaacctgccgtagaaccgaagagatatgacacgcttccatctctcaaaggaagaatcccttcagggttgcgtttccagtctagacacgtataacggcacaagtgtctctcaccaaatgggttatatctcaaatgtgatctaaggatggaaagcccagaatctaggcctattaatattccggagtatacgtagccggctaacgttaacaaccggtacctctagaactatagctagcatgcgcaaatttaaagcgctgatatcgatcgcgcgcagatccatatatagggcccgggttataattacctcaggtcgacgtcccatggccattcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgcc